(19) United States
(12) Patent Application Publication
(10) Pub. No.: US 2019/0150790 A1
(43) Pub. Date: May 23, 2019

(54) ENDOSCOPE SYSTEM

(71) Applicant: HOYA CORPORATION, Tokyo (JP)

(72) Inventors: Shinya SHIMOTASHIRO, Tokyo (JP); Shotaro KOBAYASHI, Tokyo (JP); Yoshihiro HAYASHI, Tokyo (JP)

(73) Assignee: HOYA CORPORATION, Tokyo (JP)

(21) Appl. No.: 16/098,043

(22) PCT Filed: Jul. 21, 2017
(86) PCT No.: PCT/JP2017/026584

§ 371 Date: Oct. 31, 2018

(30) Foreign Application Priority Data
Jul. 21, 2016 (JP) 2016-143505

(51) Int. Cl. A61B 5/103; A61B 1/06; A61B 1/00

(52) U.S. Cl. CPC A61B 5/1032; A61B 1/00006; A61B 1/0638

(57) ABSTRACT

An endoscope system includes: a light source that emits a first light; a rotating plate in which a first light transmitting region that allows the first light to pass therethrough, and a second light transmitting region that extracts a second light that is in at least one specific wavelength region from the first light, the first light transmitting region being configured to reduce a difference in amount between the first light that passes through the first light transmitting region and the second light extracted by the second light transmitting region; a rotation drive unit that inserts the first light transmitting region and the second light transmitting region one after the other into an optical path of the first light from the light source by rotating the rotating plate; a shift drive unit that shifts the rotating plate; and a control unit that controls the shift drive unit.

### TECHNICAL FIELD

The present invention relates to an endoscope system that is capable of switching between different types of illumination light that illuminate a subject such as a lesion site.

### BACKGROUND ART

In the field of medical devices, there is a known endoscopic system that facilitates diagnosis of a lesion site by allowing observations using illumination light in different wavelength bands with different properties to be performed at the same time. For example, Patent Document 1 discloses a specific example of a configuration of an endoscope system that can perform a normal light observation and a special light observation at the same time, as an example of such an endoscope system.

Here, a special light observation is an observation that allows an operator to identify various lesions by generating an image showing the distribution of biomolecules in biological tissue, and a special light observation function is a significantly important specification from among the product specifications of the endoscope system.

A light source unit of the endoscope system disclosed in Patent Document 1 is provided with a rotating filter in which a normal light passing area that allows normal light to pass and a special light filter area that allows special light to pass are arranged along the circumference of a circle. By driving the rotating filter to rotate, and irradiating the subject with normal light and special light one after the other to form an image, it is possible to display a normal observation image and a special light observation image on a monitor screen at the same time. Also, in the endoscope system disclosed in the Patent Document 1, the normal light passing area of the rotating filter is formed as a metal mesh light attenuation portion, is thus configured to match the amount of special light and the amount of normal light.

### PRIOR ART DOCUMENT

### Patent Document

Patent Document 1: JP 2011-200377A

### SUMMARY OF INVENTION

### Technical Problem

In recent years, there has been demand for, for example, calculating biological information such as the degree of oxygen saturation or the like of hemoglobin (as well as an evaluation value of a lesion) to be used as diagnostic aid, by employing a configuration in which the rotating filter is used to acquire a normal observation image and a special light observation image at the same time, to use image information acquired using normal light in addition to image information acquired using special light. Therefore, it is envisioned that a configuration that makes it possible to acquire a normal observation image and a special light observation image at the same time, and that does not change the amount of special light and the amount of normal light, which affects the calculation of biological information, is an important product specification that will be further required in the future as a specification of an endoscope system, from the viewpoint of improving the accuracy of diagnosis and calculation of the evaluation value of a lesion.

However, the amount of special light and the amount of normal light may change due to a positional error occurring when the rotating filter is entered into, or retracted from, an optical path extending from the light source, which results in a problem in which the accuracy of diagnosis or the evaluation value of a lesion degrades.

The present invention has been made in view of the above-described situation, and aims to provide an endoscope system that is capable of emitting a first light and a second light in different wavelength regions, and is suitable for keeping the ratio between the amount of the first light and the amount of the second light within a reference range.

### Solution to Problem

An endoscope system according to an embodiment of the present invention includes the following aspects.

(1):

An endoscope system including:

a light source that emits a first light;

a rotating plate in which a first light transmitting region that allows the first light to pass therethrough, and a second light transmitting region that extracts a second light that is in at least one specific wavelength region from the first light, are arranged in a predetermined direction, the first light transmitting region being configured to reduce a difference in the amount of light between the first light that passes through the first light transmitting region and the second light extracted by the second light transmitting region;

a rotation drive unit that inserts the first light transmitting region and the second light transmitting region one after the other into an optical path of the first light from the light source by rotating the rotating plate;

a shift drive unit that shifts the rotating plate in a direction that intersects the optical path from the light source; and

a control unit that controls the shift drive unit such that a ratio between the amount of light that has passed through the second light transmitting region and the amount of light that has passed through the first light transmitting region is within a reference range.

Preferably, the control unit generates a control signal that controls the shift drive unit such that the ratio between the amount of light that has passed through the second light transmitting region and the amount of light that has passed through the first light transmitting region is within the reference range, and transmits the control signal to the shift drive unit via a signal line.

(2):

An endoscope system including:

a light source that is configured to emit a first light;

a rotating plate that is provided with a first light transmitting region that allows the first light to pass therethrough and a second light transmitting region that extracts a second light that is in at least one specific wavelength region from the first light, and is configured to position the first light transmitting region and the second light transmitting region one after the other in an optical path of the first light to generate the first light and the second light one after the other;

a shift drive unit configured to shift the rotating plate in a direction that intersects the optical path of the first light; and

a control unit configured to control the shift drive unit such that a ratio between the amount of light that has passed through the second light transmitting region and the amount of light that has passed through the first light transmitting region is within a reference range.

Preferably, the control unit generates a control signal that controls the shift drive unit such that the ratio between the amount of light that has passed through the second light transmitting region and the amount of light that has passed through the first light transmitting region is within the reference range, and transmits the control signal to the shift drive unit via a signal line.

(3):

The endoscope system according to (1) or (2), wherein the control unit holds, in advance, a parameter for correcting the amount of displacement that is caused by the shift drive unit, between an actual position to which the rotating plate is shifted and a second position when the control unit controls the shift drive unit to shift the rotating plate from a first position to the second position, which is a target position, and controls the shift drive unit based on the parameter.

Alternatively, the endoscope system according to (1) or (2), wherein the control unit holds, in advance, a parameter for correcting the amount of displacement from a target position, which occurs when the control unit controls the shift drive unit to shift the rotating plate from a predetermined position to the target position, and controls the shift drive unit based on the parameter.

Preferably, the control unit generates a control signal that is based on the parameter, and transmits the control signal to the shift drive unit via a signal line.

(4):

The endoscope system according to any one of (1) to (3),

wherein the control unit controls the shift drive unit such that a position in the optical path into which the first light transmitting region of the rotating plate is inserted by the rotation drive unit is determined with reference to a peak position at which the light intensity of the first light from the light source is at its maximum.

Alternatively, the endoscope system according to any one of (1) to (3), wherein the control unit controls the shift drive unit such that a position in the optical path into which the first light transmitting region of the rotating plate is inserted is positioned within a predetermined range of a peak position of light from the light source.

Preferably, the control unit generates a control signal such that a position in the optical path into which the first light transmitting region of the rotating plate is inserted by the rotation drive unit is determined with reference to a peak position at which the light intensity of the first light from the light source is at its maximum, and transmits the control signal to the shift drive unit via a signal line.

(5):

The endoscope system according to any one of (1) to (4),

wherein the first light has a light intensity distribution,

a cross section of a luminous flux of the first light when entering the first light transmitting region and the second light transmitting region is larger than an incident surface of the first light transmitting region and an incident surface of the second light transmitting region, respectively, a portion of the luminous flux of the first light enters the first light transmitting region and the second light transmitting region, and the remaining portion of the luminous flux does not enter the first light transmitting region or the second light transmitting region,

the control unit controls the shift drive unit such that a portion of the luminous flux of the first light that enters at least one of the first light transmitting region and the second light transmitting region includes a peak position of the light intensity distribution.

Preferably, the control unit generates a control signal such that a portion of the luminous flux of the first light that enters at least one of the first light transmitting region and the second light transmitting region includes a peak position of the light intensity distribution, and transmits the control signal to the shift drive unit via a signal line.

(6):

The endoscope system according to any one of (1) to (5),

wherein, when shifting the rotating plate between a first position and a second position by controlling the shift drive unit, the control unit changes a drive amount of the shift drive unit according to a shift direction of the rotating plate.

Alternatively, the endoscope system according to any one of (1) to (5),

wherein, when moving the rotating plate from a predetermined position to a target position by controlling the shift drive unit, the control unit changes a drive amount of the shift drive unit when moving the rotating plate from the predetermined position to the target position according to a moving direction of the rotating plate, thereby performing control such that the first light transmitting region is located within a predetermined range of a peak position of light from the light source.

Preferably, when shifting the rotating plate between the first position and the second position by controlling the shift drive unit, the control unit generates a control signal such that a drive amount of the shift drive unit is changed according to the shift direction of the rotating plate, and transmits the control signal to the shift drive unit.

(7):

The endoscope system according to any one of (1) to (5),

wherein the control unit controls the shift drive unit such that a shift direction of the rotating plate is constant when the rotating plate is caused to enter and stop in the optical path by the shift drive unit.

Alternatively, the endoscope system according to any one of (1) to (5),

wherein the control unit drives the shift drive unit such that an entering direction of the first light transmitting region of the rotating plate is constant when the first light transmitting region is caused to enter the optical path by the shift drive unit, thereby positioning the first light transmitting region within a predetermined range of a peak position of light from the light source.

Preferably, the control unit generates a control signal such that a shift direction of the rotating plate is constant when the rotating plate is caused to enter and stop in the optical path by the shift drive unit, and transmits the control signal to the shift drive unit via a signal line.

(8):

The endoscope system according to (7),

wherein, when shifting the rotating plate from a first position toward a second position, the control unit shifts the rotating plate beyond the second position from the first position, and thereafter reverses the shift direction of the rotating plate and shifts the rotating plate to the second position.

Alternatively, the endoscope system according to (7),

wherein, when an initial moving direction in which the rotating plate is moved from a current position toward a target position is different from the entering direction that is fixed, the control unit moves the rotating plate beyond the target position by a predetermined distance in the initial moving direction, and thereafter moves the rotating plate to the target position in a direction opposite the entering direction.

Preferably, when shifting the rotating plate from a first position toward a second position, the control unit generates a control signal such that the rotating plate is shifted beyond the second position from the first position, and thereafter the shift direction of the rotating plate is reversed and the rotating plate is shifted to the second position, and transmits control signal to the shift drive unit via a signal line.

(9):

The endoscope system according to any one of (1) to (7),

wherein the control unit controls the shift drive unit based on information regarding mechanical tolerances of the shift drive unit.

Preferably, the control unit generates a control signal for controlling the shift drive unit based on information regarding mechanical tolerances of the shift drive unit, and transmits the control signal to the shift drive unit via a signal line.

(10):

The endoscope system according to any one of (1) to (9),

wherein the rotating plate is configured such that the second light transmitting region and the first light transmitting region have different widths in a radial direction.

(11):

The endoscope system according to (10),

wherein a wavelength band of the second light is narrower than a wavelength band of the first light, and

a width of the second light transmitting region in a radial direction is larger than a width of the first light transmitting region in the radial direction.

(12):

The endoscope system according to any one of (1) to (11),

wherein the light source is a lamp that emits white light, which is the first light.

(13):

The endoscope system according to any one of (1) to (12),

wherein the control unit generates, based on a ratio between a value of image data of a color component included in image data obtained by imaging biological tissue illuminated by the first light and a value of image data of a color component included in image data obtained by imaging the subject illuminated by the second light, information regarding a state of the biological tissue.

Preferably, the control unit transmits the information to a display apparatus to display the information on the display apparatus.

### Advantageous Effects of Invention

As described above, with the endoscope system above, it is possible to provide an endoscope system that is capable of emitting a first light and a second light that are in different wavelength regions, and is suitable for keeping the ratio between the amount of the first light and the amount of the second light within a reference range.

### BRIEF DESCRIPTION OF DRAWINGS

FIG. 1 shows the Q band absorption spectrum of hemoglobin.

FIG. 2 is a diagram showing results of simulation of the spectral characteristics of biological tissue.

FIG. 3 includes graphs showing the correlation between various parameters and biological information.

FIG. 4 includes graphs showing the correlation between various parameters and biological information.

FIG. 5 includes graphs showing the correlation between various parameters and biological information.

FIG. 6 is a block diagram showing an example of an endoscope system according to an embodiment.

FIG. 7 is a diagram showing an example of the transmission spectrum of color filters included in an image sensor of the endoscope system according to the embodiment.

FIG. 8 is an external view of an example of a rotating filter of the endoscope system according to the embodiment.

FIG. 9 is a flowchart illustrating an example of spectral analysis processing that is performed by the endoscope system according to the embodiment.

FIG. 10 is a block diagram showing an example of a configuration of a shift drive mechanism in the endoscope system according to the embodiment.

FIG. 11 is a diagram illustrating variation of the stop position of the rotating filter occurring due to manufacturing tolerances of the mechanical mechanism.

FIG. 12 is a diagram illustrating the relationship between the distribution of intensities of white light from a light source and the position of a slit.

FIG. 13 is a diagram illustrating a state in which the position at which a rack gear of the shift drive mechanism in the endoscope system according to the embodiment stops in a forward movement is caused to accurately coincide with that in a backward movement.

FIG. 14 is a flowchart showing an example of control of the stop position of the rotating filter in the endoscope system according to the embodiment.

FIG. 15 is a diagram illustrating an operating principle regarding an example of control of the stop position of the rotating filter in the endoscope system according to the embodiment.

FIG. 16 is a flowchart showing an example of control of the stop position of the rotating filter in the endoscope system according to the embodiment.

FIG. 17 illustrates brightness adjustment for a normal observation image, which is performed by controlling a shift drive function of the endoscope system according to the embodiment.

### DESCRIPTION OF EMBODIMENTS

Hereinafter, an embodiment of the present invention will be described with reference to the drawings.

An endoscope system according to the embodiment of the present invention described below is an apparatus for quantitatively analyzing biological information of a subject (e.g., a feature amount of biological tissue such as the total hemoglobin amount or the degree of oxygen saturation) based a plurality of images of a subject captured under different types of illumination light that are in different wavelength regions, and for converting the analysis results into an image and displaying the image. The spectral characteristics of blood (i.e., the spectral characteristics of hemoglobin) have a property of continuously changing according to the total hemoglobin amount and the degree of oxygen saturation, and this property is used in the quantitative analysis of the total hemoglobin amount and the degree of oxygen saturation described below. Note that examples of parts that are to be observed using the endoscope system according to the present embodiment include respiratory organs, digestive organs, and so on. Respiratory organs include, for example, the lungs, the ears, the nose, and the throat. Digestive organs include, for example, the large intestine, the small intestine, the stomach, the esophagus, the duodenum, and the uterus.

Also, as described below, the endoscope system according to the present embodiment is configured to emit a first light and a second light that are in different wavelength regions (i.e., illumination light in different wavelength regions) one after the other. Specifically, the endoscope system according to the present embodiment includes a light source unit that emits white light, which is the first light, and a rotating plate for extracting the second light in a specific wavelength region from the white light. The present embodiment describes a rotating filter, which is an example of the rotating plate. Note that, in the present description, white light from the light source is also referred to as normal light, and light that has passed through an optical filter of the rotating filter is also referred to as special light. The configuration of the rotating filter and a configuration for driving and moving the rotating filter back and forth between a retracted position and an application position will be described later. The application position is a position at which the rotating filter allows the peak position of the luminous flux of white light to pass. The peak position indicates the maximum intensity of the distribution of light intensities.

### Spectral Characteristics of Biological Tissue and Principle of Calculation of Biological Information

Before giving a description of the detailed configuration of the endoscope system according to the embodiment of the present invention, the following describes the spectral characteristics of hemoglobin and the principle of the calculation of a feature amount of biological tissue (biological information), such as the degree of oxygen saturation, according to the embodiment of the present invention.

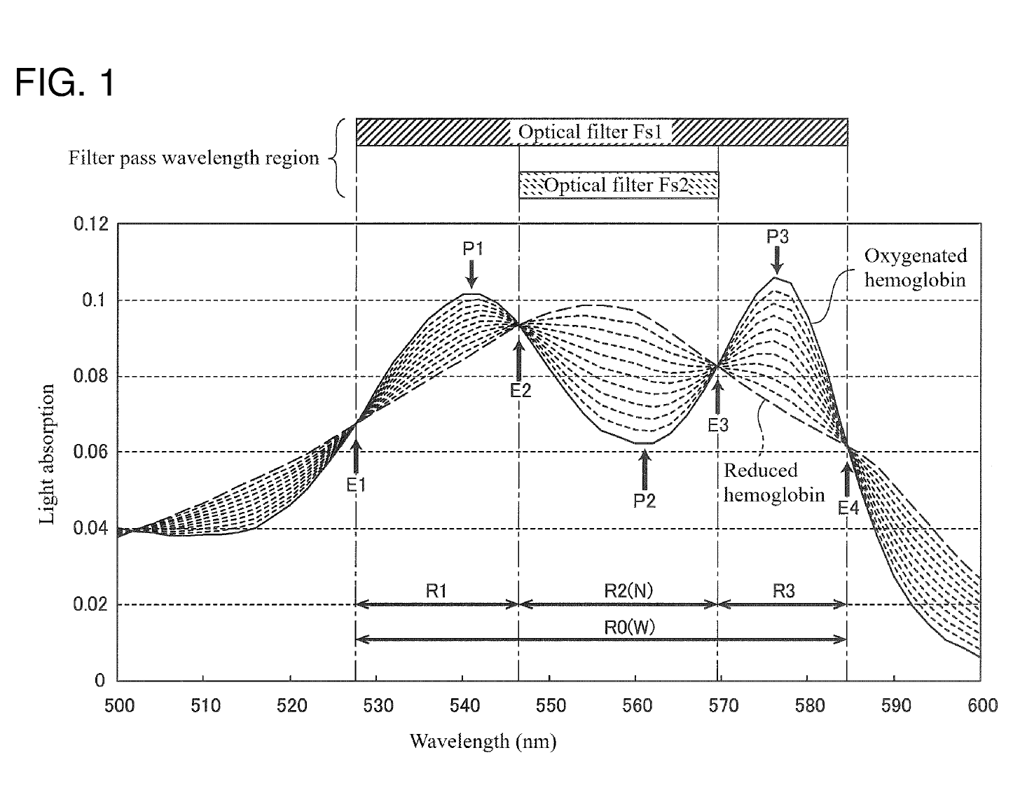

FIG. 1 shows the absorption spectrum of hemoglobin at roughly 550 nm. Hemoglobin has a strong absorption band at roughly 550 nm that is called the Q band and derives from porphyrin. The absorption spectrum of hemoglobin varies according to the degree of oxygen saturation. The degree of oxygen saturation is the percentage of oxygenated hemoglobin HbO in the total amount of hemoglobin. The solid line waveform in FIG. 1 is the absorption spectrum of oxygenated hemoglobin HbO in the case where the degree of oxygen saturation is 100% (i.e., the absorption spectrum of oxygenated hemoglobin), and the long dashed line waveform is the absorption spectrum in the case where the degree of oxygen saturation is 0% (i.e., the absorption spectrum of reduced hemoglobin Hb), that is to say the absorption spectrum of reduced hemoglobin Hb. Also, the short dashed lines are the absorption spectrums of hemoglobin (mixture of oxygenated hemoglobin HbO and reduced hemoglobin Hb) at intermediate degrees of oxygen saturation (10, 20, 30, . . . 90%).

As shown in FIG. 1, in the Q band, oxygenated hemoglobin HbO and reduced hemoglobin Hb have mutually different peak wavelengths. Specifically, oxygenated hemoglobin HbO has an absorption peak P1 at a wavelength of roughly 542 nm and an absorption peak P3 at a wavelength of roughly 576 nm. On the other hand, reduced hemoglobin Hb has an absorption peak P2 at roughly 556 nm. FIG. 1 shows a two-component absorption spectrum in which the sum of the concentrations of the respective components (oxygenated hemoglobin HbO and reduced hemoglobin Hb) is constant, and therefore isosbestic points E1, E2, E3, and E4, at which the absorption is constant regardless of the concentrations of the respective components (i.e., the degree of oxygen saturation), appear in the spectrum. In the following description, the wavelength region sandwiched between the isosbestic points E1 and E2 will be called a wavelength region R1, the wavelength region sandwiched between the isosbestic points E2 and E3 will be called a wavelength region R2, and the wavelength region sandwiched between the isosbestic points E3 and E4 will be called a wavelength region R3. Also, the wavelength region sandwiched between the isosbestic points E1 and E4 (i.e., the combination of the wavelength regions R1, R2, and R3) will be called a wavelength region R0. Also, in the following description, the wavelength region R2 is also called the N band (Narrow-band), and the wavelength region R0 is also called the W band (Wide-band).

As shown in FIG. 1, in the wavelength regions between adjacent isosbestic points, the absorption of hemoglobin increases or decreases linearly relative to the degree of oxygen saturation.

Specifically, absorbances A_{R1} and A_{R3} of hemoglobin in the wavelength regions R1 and R3 (the values of integral of the wavelength regions R1 and R3) linearly increase relative to the concentration of oxygenated hemoglobin. Also, absorbance A_{R2} of hemoglobin in the wavelength region R2 linearly increases relative to the concentration of reduced hemoglobin.

Here, the degree of oxygen saturation is defined by Expression 1 below.

$$Sat = \frac{[ HbO ]}{[ Hb ] + [ HbO ]} \qquad Expression 1$$

where

Sat: degree of oxygen saturation

[Hb]: concentration of reduced hemoglobin

[HbO]: concentration of oxygenated hemoglobin

[Hb]+[HbO]: total hemoglobin amount (tHb)

Also, Expression 2 and Expression 3 that express the concentrations of oxygenated hemoglobin HbO and reduced hemoglobin are obtained from Expression 1.

[HbO]=Sat·([Hb]+[HbO]) Expression 2

[Hb]=(1−Sat)·([Hb]+[HbO]) Expression 3

Accordingly, the absorbances A_{R1}, A_{R2}, and A_{R3} of hemoglobin are characteristic values that are dependent on both the degree of oxygen saturation and the total hemoglobin amount.

Also, through research by the applicant of this patent application, it was found that the absorbance A_{R0} of hemoglobin in the wavelength region R0, which is made up of the wavelength regions R1, R2, and R3, (the value of integral of the wavelength region R0), is a value that is not dependent on the degree of oxygen saturation, but is determined by the total hemoglobin amount.

Accordingly, the total hemoglobin amount can be determined based on the absorbance A_{R0}. Also, the degree of oxygen saturation Sat can be determined based on the absorbances A_{R1}, A_{R2}, and A_{R3}, and the total hemoglobin amount determined based on the absorbance A_{R0}. Note that as shown in FIG. 1, the amount of variation of absorbance according to the degree of oxygen saturation in the wavelength regions R1, R2, and R3 (i.e., the area of the region enclosed by the solid-line waveform and the long-dash waveform) is the largest in the wavelength region R2, and the absorbance A_{R2} of the wavelength region R2 is the characteristic amount that is most sensitive to the degree of oxygen saturation. In the embodiment described later, the degree of oxygen saturation is also determined using light in the wavelength region R2 (N band).

Next, the influence of scattering on the spectral characteristics of biological tissue will be described.

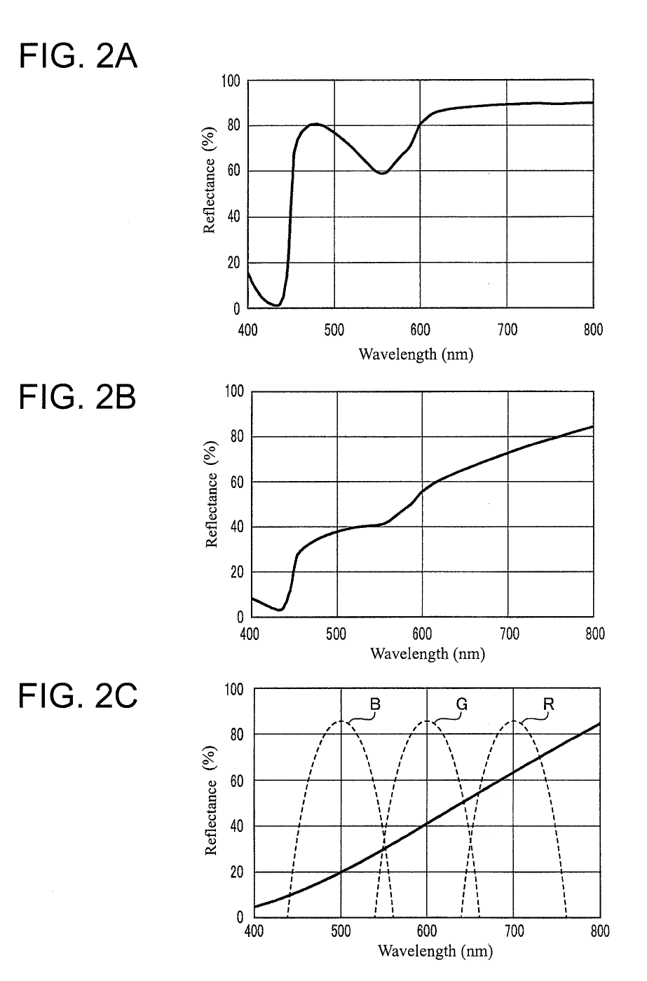

FIG. 2 shows examples of a reflection spectrum that indicates the spectral characteristics of biological tissue in the visible light region obtained by simulation calculation, and shows the influence of light scattering on spectral characteristics. In the graphs in FIG. 2, the horizontal axis indicates the wavelength, and the vertical axis indicates the reflectance. The reflection spectrum of biological tissue such as a digestive track wall is influenced by not only the absorption wavelength characteristics of the components that make up the biological tissue, specifically the absorption spectrum characteristics of oxygenated hemoglobin and reduced hemoglobin, but also the wavelength characteristics of light scattering by biological tissue. FIG. 2(a) shows the reflection spectrum in the case of no light scattering whatsoever, FIG. 2(c) shows the reflection spectrum in the case where there is no absorption whatsoever by hemoglobin, and light scattering occurs, and FIG. 2(b) shows the reflection spectrum in the case where the contribution of light scattering by biological tissue (light attenuation caused by scattering) and the contribution of hemoglobin absorption (light attenuation caused by absorption) on the reflection spectrum are approximately the same.

As shown in FIG. 2, the biological tissue spectral characteristics vary according to the intensity of light scattering, and therefore if biological information such as the degree of oxygen saturation is calculated based on the biological tissue spectral characteristics without giving consideration to the degree of strength of light scattering, the biological information can change in value according to the intensity of light scattering. In other words, if the biological tissue spectral characteristics (e.g., reflectance in the wavelength region R2) are used as-is to calculate the biological information, a calculation result that contains errors arising from light scattering will be obtained. In order to obtain a precise analysis result, it is necessary to correct the errors arising from light scattering.

Methods of correcting error arising from light scattering include a method of correcting error after calculating biological information such as the degree of oxygen saturation Sat based on biological tissue spectral characteristics, and a method of generating an intermediate parameter that is not dependent on light scattering based on biological tissue spectral characteristics, removing the component that is dependent on light scattering at the stage of generating the intermediate parameter, and then calculating biological information based on the correlation relationship between the intermediate parameter and the biological information, that is to say a biological tissue feature amount. In the present embodiment, the latter method is used to acquire biological information that does not contain error arising from light scattering. In order to realize this method, the inventors of the present invention searched for a parameter that has high sensitivity to (is highly correlated with) biological information that is to be acquired, specifically the total hemoglobin amount and the degree of oxygen saturation that are biological tissue feature amounts, and is also unlikely to produce an error arising from light scattering, i.e., unlikely to change according to the strength of light scattering. Hereinafter, the fact of being unlikely to change according to the strength of light scattering is also referred to as having no sensitivity to light scattering.

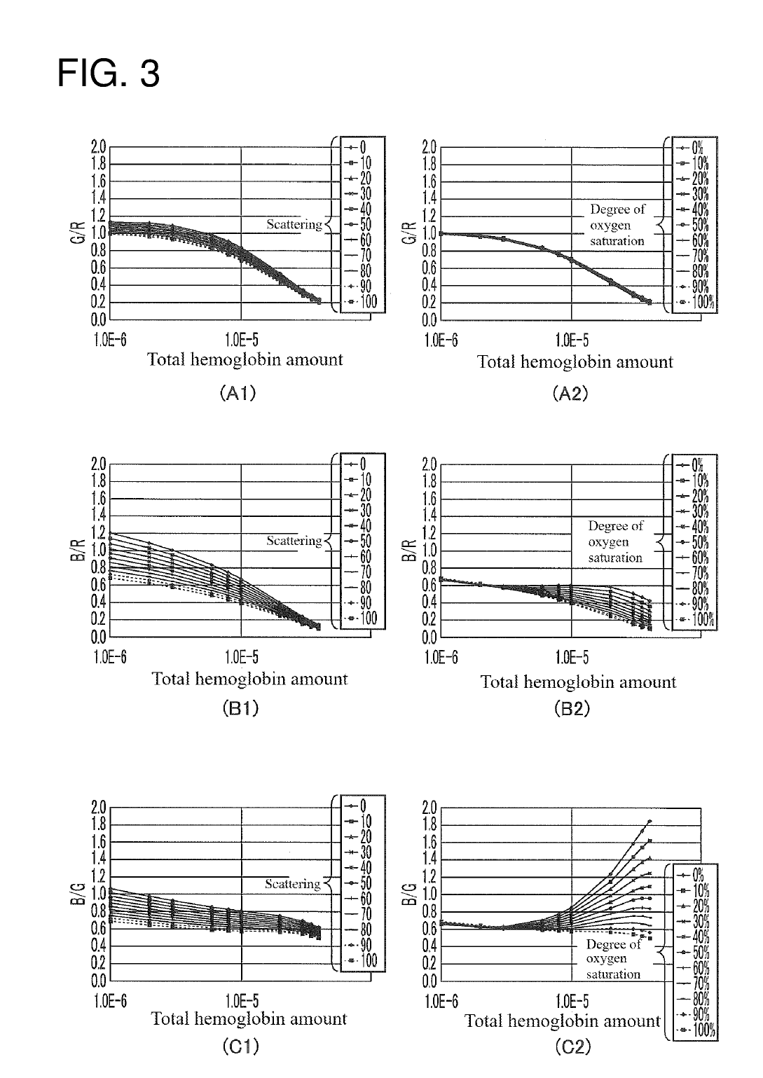

FIGS. 3 to 5 are graphs showing examples of the correlation between various parameters that can be acquired from endoscopic image data and the total hemoglobin amount tHb and the degree of oxygen saturation Sat, and these graphs are plots of simulation results of the parameters. The horizontal axis in the graphs indicates the total hemoglobin amount tHb, and the vertical axis indicates parameter values. Also, Table 1 is an organized arrangement of elements in the graphs of FIGS. 3 to 5.

Note that “sensitivity” in Table 1 is indicated using one to three stars representing the sensitivity (i.e., magnitude of variation range) of the parameters relative to change in the total hemoglobin amount tHb, the intensity of light scattering, and the degree of oxygen saturation Sat, as interpreted from the graphs of FIGS. 3 to 5. A larger number of stars indicates higher parameter sensitivity, that is to say indicates a larger variation range.

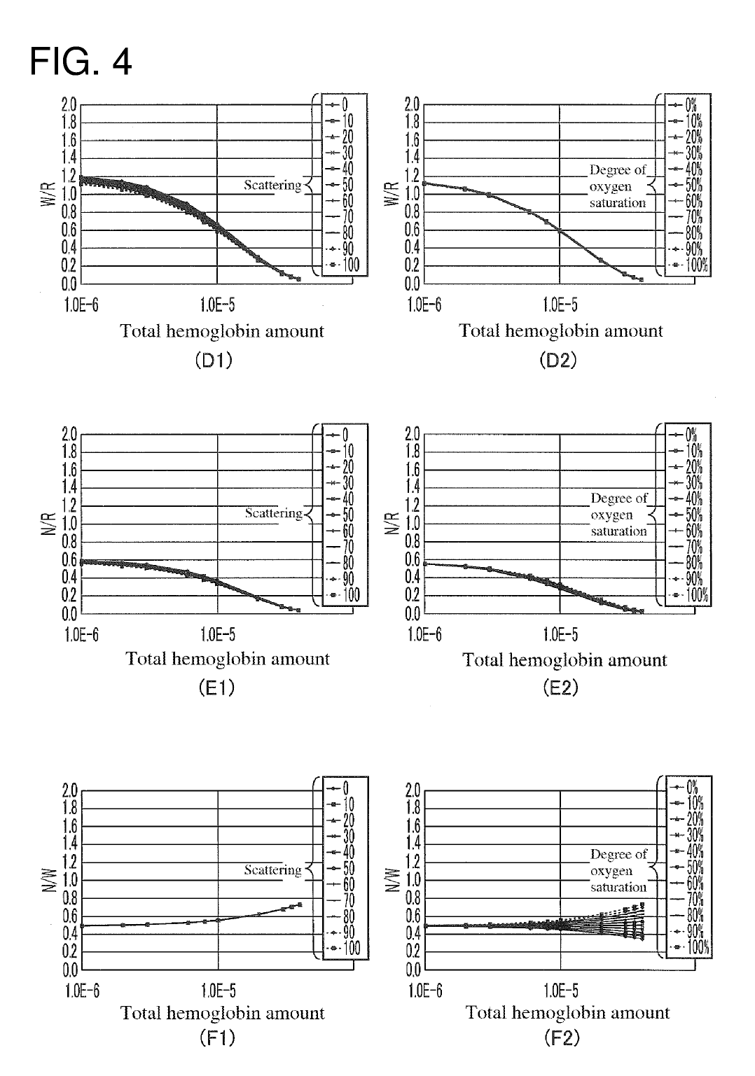

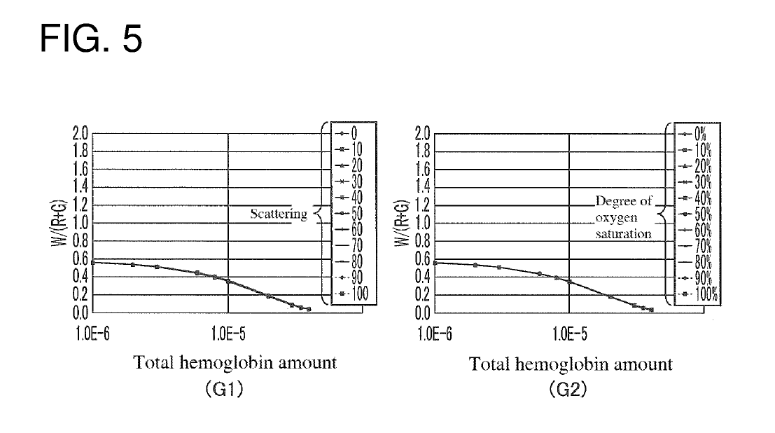

TABLE 1

| | | | Setting | | Sensitivity | | |
|---|---|---|---|---|---|---|---|
| | | | | Degree of | | | Degree of |
| Graph | | Parameter | Contribution of scattering | oxygen saturation | Total hemoglobin amount | Scattering | oxy. saturation |
| FIG. 3 | (A1) | G/R | 0~100 | 100% | ★★★ | ★★ | |
| | (A2) | | 0 | 0~100% | | | ★ |
| | (B1) | B/R | 0~100 | 100% | ★★ | ★★★ | |
| | (B2) | | 0 | 0~100% | | | ★★ |
| | (C1) | B/G | 0~100 | 100% | ★★ | ★★ | |
| | (C2) | | 0 | 0~100% | | | ★★★ |
| FIG. 4 | (D1) | W/R | 0~100 | 100% | ★★★ | ★ | |
| | (D2) | | 0 | 0~100% | | | ★ |
| | (E1) | N/R | 0~100 | 100% | ★★ | ★ | |
| | (E2) | | 0 | 0~100% | | | ★ |
| | (F1) | N/W | 0~100 | 100% | ★ | ★ | |
| | (F2) | | 0 | 0~100% | | | ★★ |
| FIG. 5 | (G1) | W/(R + G) | 0~100 | 100% | ★★ | ★ | |
| | (G2) | | 0 | 0~100% | | | ★ |

Graphs (A1) and (A2) in FIG. 3 are graphs plotting simulation results for the parameter “G/R”. “G” is the pixel value of G pixels (pixels provided with the green G color filter) obtained by normal observation with use of white light as illumination light for the biological tissue. Also, “R” is the pixel value of R pixels (pixels provided with the red R color filter) obtained by normal observation. The parameter “G/R” is the result of dividing the pixel value G by the pixel value R, each obtained by normal observation. Normal observation refers to imaging biological tissue using white light, and acquiring an image that has an R component, a G component, and a B component in the RGB color space.

Note that in the present specification, pixel values are not limited to pixel values of an imaging signal (so-called RAW data) from an image sensor that includes an RGB primary color filter, and also include pixel values of image data obtained by performing various types of image processing such as demosaic processing (interpolation processing) and linear matrix processing on an imaging signal. For example, later-described processing can also be performed using R pixel values, G pixel values, and B pixel values that are the R values, G values, and B values of pixels included in image data that has an R component, a G component, and a B component in the RGB color space obtained by performing demosaic processing and color space conversion processing on an imaging signal from an image sensor that includes a complementary color filter.

Graphs (B1) and (B2) in FIG. 3 are graphs plotting simulation results for the parameter “B/R”. Also, “B” is the pixel value of B pixels (pixels provided with the blue B color filter) obtained by normal observation performed using white light. The parameter “B/R” is the result of dividing the pixel value B by the pixel value R, each obtained by normal observation.

Graphs (C1) and (C2) in FIG. 3 are graphs plotting simulation results for the parameter “B/G”. The parameter “B/G” is the result of dividing the pixel value B by the pixel value G, each obtained by normal observation.

Graphs (D1) and (D2) in FIG. 4 are graphs plotting simulation results for the parameter “W/R”. “W” is the pixel value of G pixels obtained by special observation performed using illumination light in the wavelength region R0 (W band) shown in FIG. 1. Note that as will be described later, the wavelength region R0 is included in a wavelength region in which G pixels of the image sensor have sensitivity. The parameter “W/R” is the result of the pixel value W of G pixels obtained by special observation performed using illumination light in the W band being divided by the pixel value R obtained by normal observation.

Graphs (E1) and (E2) in FIG. 4 are graphs plotting simulation results for the parameter “N/R”. “N” is the pixel value of G pixels obtained by special observation performed using illumination light in the wavelength region R2 (N band) shown in FIG. 1. The parameter “N/R” is the result of the pixel value N of G pixels obtained by special observation performed using illumination light in the N band being divided by the pixel value R obtained by normal observation.

Graphs (F1) and (F2) in FIG. 4 are graphs plotting simulation results for the parameter “N/W”. The parameter “N/W” is the result of the pixel value N of G pixels obtained by special observation performed using illumination light in the N band being divided by the pixel value W of G pixels obtained by special observation performed using illumination light in the W band.

Graphs (G1) and (G2) in FIG. 5 are graphs plotting simulation results for the parameter “W/(R+G)”. The parameter “W/(R+G)” is the result of the pixel value W of G pixels obtained by special observation performed using illumination light in the W band being divided by the sum “R+G” of the pixel value R of R pixels and the pixel value G of G pixels obtained by normal observation performed using white light as illumination light.

Also, the graphs (A1), (B1), (C1), (D1), (E1), (F1), and (G1) on the left side in FIGS. 3 to 5 are graphs in which the degree of oxygen saturation is fixed at 100%, and the contribution of light scattering (parameter indicating the intensity of light scattering) is varied between 0 and 100 in units of 10 and plotted in an overlapped manner. Based on these graphs, it is possible to find out the degree of sensitivity of the parameters to light scattering.

Also, the graphs (A2), (B2), (C2), (D2), (E2), (F2), and (G2) on the right side in FIGS. 3 to 5 are graphs in which the contribution of scattering is set to 0, and the degree of oxygen saturation is varied between 0 and 100% in units of 10% and plotted in an overlapped manner. Based on these graphs, it is possible to find out the degree of sensitivity of the parameters to the degree of oxygen saturation.

As shown in Table 1 and the graphs (D1) and (D2) in FIG. 4, the parameter “W/R” has high sensitivity to the total hemoglobin amount, but has almost no sensitivity to light scattering or the degree of oxygen saturation. For this reason, the value of the total hemoglobin amount is uniquely determined by the value of the parameter “W/R”. In other words, an accurate total hemoglobin amount that is not dependent on light scattering or degree of oxygen saturation can be obtained based on the value of the parameter “W/R” obtained from image data and the quantitative relationship between the total hemoglobin amount and the parameter “W/R” shown in the graphs (D1) and (D2).

Also, as shown in Table 1 and the graphs (F1) and (F2) in FIG. 4, the parameter “N/W” has high sensitivity to the degree of oxygen saturation, but has almost no sensitivity to light scattering. For this reason, if the total hemoglobin amount is known, the value of the degree of oxygen saturation can be uniquely determined based on the value of the parameter “N/W” according to the graph (F2). Specifically, if the plotted point in the graph (F2) that most closely conforms to the numerical value pair of the value of the total hemoglobin amount and the value of the parameter “N/W” obtained from the pixel values is selected, the value of the degree of oxygen saturation corresponding to the plotted point is used as the degree of oxygen saturation of the biological tissue appearing at that pixel. Note that the value of the total hemoglobin amount is obtained based on the value of the parameter “W/R” obtained from the image data and the relationship between the total hemoglobin amount and the parameter “W/R” indicated in the graphs (D1) and (D2).

Also, as shown in Table 1 and the graphs (G1) and (G2) in FIG. 5, similarly to the parameter “W/R” described above, the parameter “W/(R+G)” has sensitivity to the total hemoglobin amount, but has almost no sensitivity to light scattering or degree of oxygen saturation, and therefore an accurate value of the total hemoglobin amount that is not dependent on light scattering or the degree of oxygen saturation is obtained based on the quantitative relationship between the total hemoglobin amount and the parameter “W/(R+G)” shown in the graphs (G1) and (G2).

As described above, by performing simple calculation using the relationships shown in the graphs (D1) and (D2) or the graphs (G1) and (G2), along with the relationship shown in the graph (F2) or (C2), it is possible to obtain accurate values for the total hemoglobin amount and the degree of oxygen saturation that contain almost no error arising from scattering.

### Configuration of Endoscope System

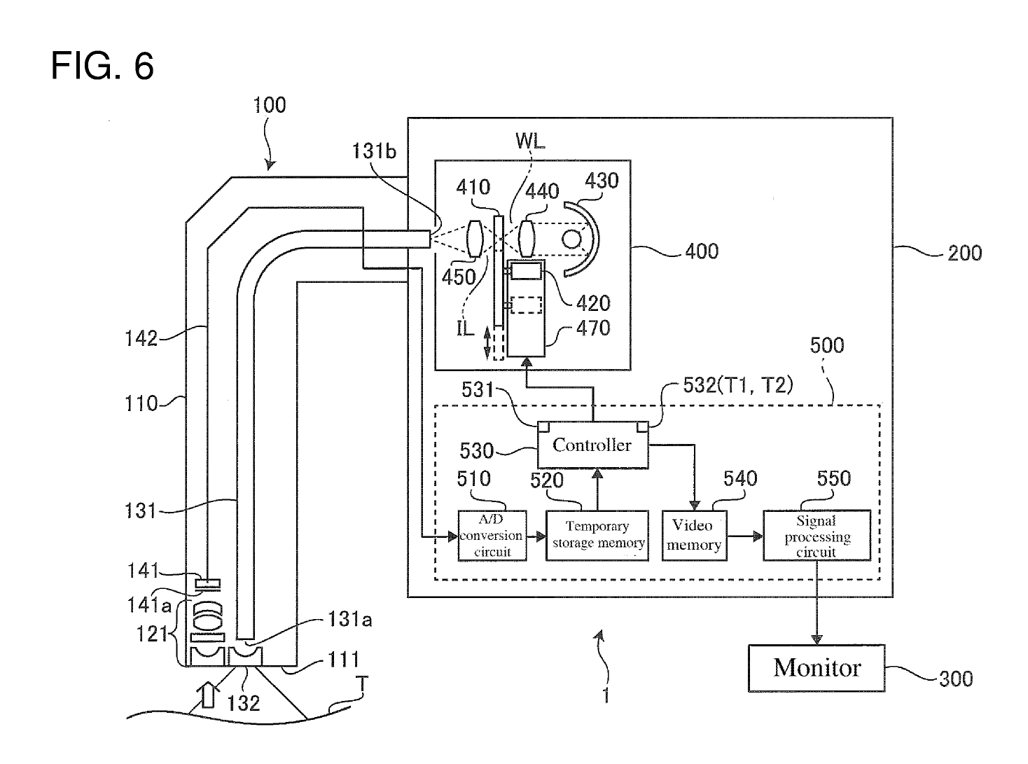

FIG. 6 is a block diagram showing an endoscope system 1 according to the embodiment of the present invention. The endoscope system 1 according to the present embodiment includes an electronic endoscope 100, a processor 200, and a monitor 300. The electronic endoscope 100 and the monitor 300 are detachably connected to the processor 200. Also, a light source unit 400 and an image processing unit 500 are built into the processor 200. Although the light source unit 400 is built into the processor 200 in the present embodiment, the light source unit 400 need not be built into the processor 200. For example, the light source unit 400 may be configured as a light source apparatus that is separate from the processor.

The electronic endoscope 100 has an insertion tube 110 for insertion into the subject's body. The electronic endoscope 100 is internally provided with a light guide 131 that extends over approximately the entire length thereof. One end portion (distal end portion 131a) of the light guide 131 is arranged in the distal end portion of the insertion tube 110 (insertion tube distal end portion 111), and the other end portion (base end portion 131b) of the light guide 131 is connected to the processor 200. The processor 200 includes a light source unit 400 that includes a light source lamp 430 or the like for generating high-intensity white light WL, such as a xenon lamp. The illumination light IL generated by the light source unit 400 enters the base end 131b of the light guide 131. Light that enters the base end 131b of the light guide 131, passes through the light guide 131 and is guided to the distal end portion 131a thereof, and is then emitted from the distal end portion 131a. A light distribution lens 132 arranged opposing the distal end portion 131a of the light guide 131 is provided at the insertion tube distal end portion 111 of the electronic endoscope 100, and illumination light IL emitted from the distal end portion 131a of the light guide 131 passes through the light distribution lens 132 and illuminates biological tissue T in the vicinity of the insertion tube distal end portion 111.

Also, the insertion tube distal end portion 111 is provided with an objective optical system 121 and an image sensor 141. Part of the illumination light IL reflected or scattered by the surface of the biological tissue T (returning light) enters the objective optical system 121, is condensed, and forms an image on the light receiving surface of the image sensor 141. The image sensor 141 of the present embodiment is a CCD (Charge Coupled Device) image sensor for color image capturing, and includes a color filter 141a on its light receiving surface. Another type of image sensor such as a CMOS (Complementary Metal Oxide Semiconductor) image sensor may be used as the image sensor 141.

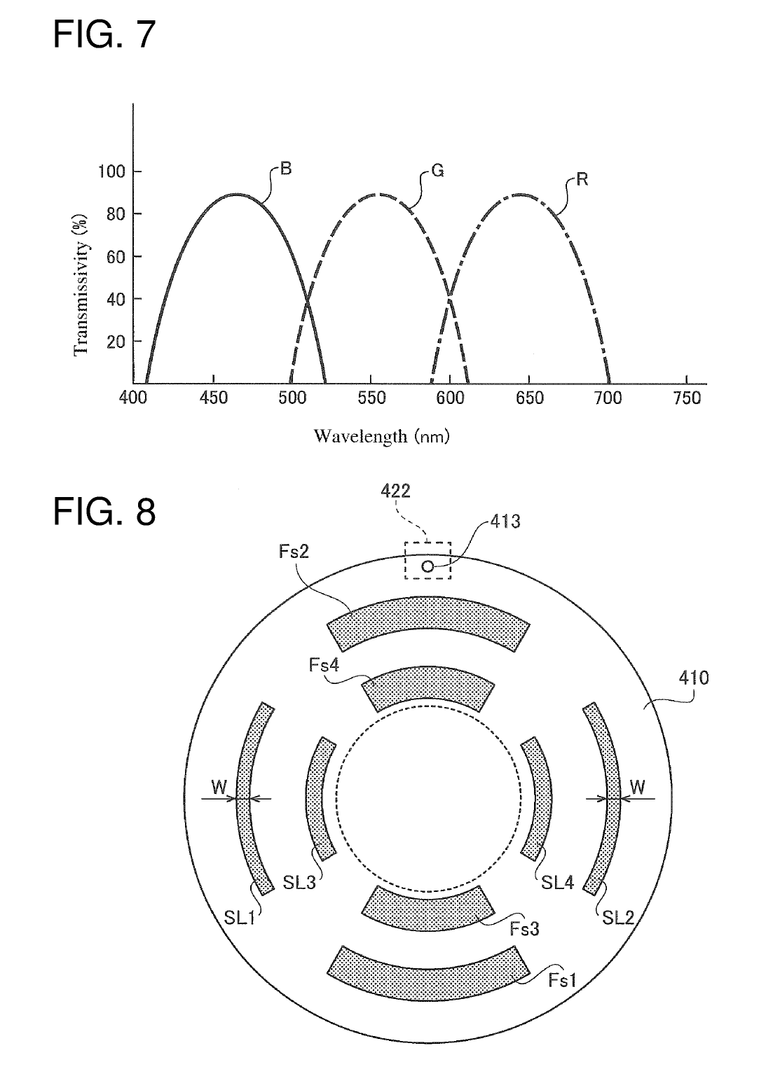

The color filter 141a includes an array of R color filters that allow red light to pass, G color filters that allow green light to pass, and B color filters that allow blue light to pass, and is a so-called on-chip filter that is formed directly on the light receiving element of the image sensor 141. The R, G, and B filters have the spectral characteristics shown in FIG. 7. That is to say, the R color filters of the present embodiment are filters that allow light with a wavelength longer than approximately 570 nm to pass, the G color filters are filters that allow light with a wavelength of approximately 470 nm to 620 nm to pass, and the B color filters are filters that allow light with a wavelength shorter than approximately 530 nm to pass.

The image sensor 141 is controlled to drive in synchronization with a signal processing unit 550 that will be described later, and periodically (e.g., at intervals of 1/30 second) outputs an imaging signal that corresponds to an image of the subject that is formed on the light receiving surface. The imaging signal output from the image sensor 141 is sent to the image processing unit 500 of the processor 200 via a cable 142.

The image processing unit 500 includes an A/D conversion circuit 510, a temporary storage memory 520, a controller 530, a video memory 540, and a signal processing circuit 550. The A/D conversion circuit 510 performs A/D conversion on an imaging signal received from the image sensor 141 of the electronic endoscope 100 via the cable 142, and outputs digital image data. The digital image data output from the A/D conversion circuit 510 is sent to and stored in the temporary storage memory 520. This digital image data includes R digital image data obtained by the light receiving elements on which the R color filters are mounted, G digital image data obtained by the light receiving elements on which the G color filters are mounted, and B digital image data obtained by the light receiving elements on which the B color filters are mounted. Note that, in the present specification, R digital image data, G digital image data, and B digital image data may also be referred to as single color image data (R single color image data, G single color image data, and B single color image data).

The controller 530 processes one or more pieces of digital image data stored in the temporary storage memory 520 to generate screen data for display on the monitor 300, and sends the screen data to the video memory 540. For example, the controller 530 generates a reflection spectrum for the biological tissue T for each pixel (x,y) based on screen data generated based on one piece of digital image data, based on screen data in which multiple pieces of digital image data are arranged side-by-side, or based on multiple pieces of digital image data, then uses the reflection spectrum to generate screen data that includes an image that shows healthy sites and lesion sites in different colors, or generate screen data that displays a graph of the reflection spectrum of the biological tissue T that corresponds to a certain pixel (x,y), and then stores the screen data in the video memory 540. The signal processing circuit 550 generates a video signal in a predetermined format (e.g., a format compliant with NTSC standards or DVI standards) based on screen data that is stored in the video memory 540, and outputs the video signal. The video signal output from the signal processing circuit 550 is received by the monitor 300. As a result, an endoscopic image or the like captured by the electronic endoscope 100 is then displayed on the monitor 300.

Note that the controller 530 generates control signals for controlling the components of the light source unit 400, and transmit the control signals to the components via signal lines.

In this way, the processor 200 includes both functionality as a video processor that processes imaging signals output from the image sensor 141 of the electronic endoscope 100, and functionality as a light source apparatus that supplies illumination light IL, which is for illuminating biological tissue T that is the imaging subject, to the light guide 131 of the electronic endoscope 100.

Besides the above-described light source 430, the light source unit 400 also includes a condensing lens 440, a rotating filter (rotating plate) 410, a filter control unit 420, and a condensing lens 450. Approximately parallel white light WL that exits from the light source 430 is condensed by the condensing lens 440, passes through the rotating filter 410, is then again condensed by the condensing lens 450, and then enters the base end 131b of the light guide 131.

The light source unit 400 also includes a shift drive mechanism (shift drive unit) 470. Due to this shift drive mechanism 470, the rotating filter 410 is movable between the application position in the optical path of white light WL and a retracted position that is out of the optical path. The details of the shift drive mechanism 470 will be described later.

White light WL that exits from the light source 430 enters the rotating filter 410. FIG. 8 is a front view of the rotating filter 410 seen from the condensing lens 450 side. In FIG. 8, for the purpose of illustration, the shift drive mechanism 470, which drives and moves the rotating filter 410 back and forth in a direction that is orthogonal to the optical path of light from the light source 430, is omitted. As shown in FIG. 8, the rotating filter 410 includes four fan-shaped optical filters (special light filters) Fs1, Fs2, Fs3, and Fs4, and four slits SL1, SL2, SL3, and SL4. The special light filter Fs1, the slit SL1, the special light filter Fs2, and the slit SL2 are arranged on the rotating filter 410's outer circumference side, at angle pitches (angle pitches of 90° in this example) corresponding to the period of imaging cycles (the frame period). Also, the special light filter Fs2, the slit SL3, the special light filter Fs4, and the slit SL4 are arranged on the rotating filter 410's inner circumference side at angle pitches (angle pitches of 90° in this example) corresponding to the period of imaging cycles (the frame period). All of the special light filters Fs1, Fs2, Fs3, and Fs4 are dielectric multilayer filters, but they may be optical filters of another type (e.g., etalon filters using a dielectric multilayer film as a reflection film). Each of the special light filters Fs1, Fs2, Fs3, and Fs4 acts to extract special light (light in a specific wavelength region). In the following description, “frame” may be replaced with “field”. In the present embodiment, the frame period and the field period are respectively 1/30 seconds and 1/60 seconds, for example.

In the above-described configuration, the rotating filter 410 is shifted by the shift drive mechanism 270 such that the special light filter Fs1, the slit SL1, the special light filter Fs2, and the slit SL2 arranged on the rotating filter unit 260's outer circumference side are positioned on the optical path of light from the light source 430, and accordingly the subject is successively irradiated with special light that passes through the special light filter Fs1, normal light that passes through the slit SL1, special light that passes through the special light filter Fs2, and normal light that passes through the slit SL2, in units of frames (or in units of fields). As shown in FIG. 8, the slits (SL1 and SL2) are different from the special light filters (Fs1 and Fs2) in width in a radial direction. Specifically, they are configured such that the width of the slits (SL1 and SL2) in a radial direction is narrower than the width of the special light filters in a radial direction. More specifically, the width w of the slits SL1 and SL2 in a radial direction is, for example, set such that the ratio between the amount of light transmitting through the slit SL1 (SL2) and the amount of light transmitting through the special light filter Fs1 and/or the special light filter Fs2 falls within a reference range. This reference range is preferably set to be constant. Thus, it is possible to perform control to, for example, limit the ratio between the brightness of a normal observation image obtained through the slit SL1 (or SL2) and the brightness of a special light observation image obtained through the special light filter Fs1 and/or the special light filter Fs2 to a constant reference range or to a constant value. Also, as a result, it is possible to keep the accuracy of a calculation of biological information such as the degree of oxygen saturation, and it is possible to prevent the occurrence of a phenomenon in which, when a normal observation image and a special light observation image are displayed at the same time, one of the images is dark, for example. Thus, it is possible to improve the accuracy of a diagnosis of a lesion site performed by an operator. Note that, in one embodiment, the reference range is preferably determined to be a range that makes it possible to keep the accuracy of calculation of biological information such as the degree of oxygen saturation of hemoglobin, for example. In one embodiment, the reference range is preferably determined for each type of biological information based on the results of a test performed in advance using samples that have known biological information. In this case, it is preferable that the endoscope system 1 is configured to hold a table of values that are respectively defined for types of biological information, as the reference range of the ratio between the amount of light passing through a slit and the amount of light passing through a special light filter.

Note that the endoscope system 1 is configured such that the operator can select the set of special light filters and slits on the rotating filter 410's outer circumference side (Fs1, SL1, Fs2, and SL2) or the set of special light filters and slits on the inner circumference side (Fs3, SL3, Fs4, and SL4) so as to be located on the optical path of light from the light source 430 by operating an operation panel (not shown) of the processor 200 according the purpose of observation. When special light observation is to be performed, the controller 530 drives and controls the shift drive mechanism 270 according to an operation input by the operator, and positions the set of special light filters and slits on the rotating filter 410's outer circumference side (Fs1, SL1, Fs2, and SL2) or the set of special light filters and slits on the inner circumference side (Fs3, SL3, Fs4, and SL4) on the optical path.

The following describes a case where the special light filters Fs1 and Fs2 on the outer circumference side are configured as optical filters for observation of the degree of oxygen saturation, for example. In addition, for the purpose of illustration, the special light filters Fs1 and Fs2 are also referred to as a “first oxygen saturation degree observation filter Fs1” and a “second oxygen saturation degree observation filter Fs2”, respectively. In this case, according to an embodiment, it is preferable that the special light filters Fs3 and Fs4 on the inner circumference side are formed as optical filters for infrared light observation.

The first oxygen saturation degree observation filter Fs1 is an optical bandpass filter that selectively allows light in the 550 nm band to pass. As shown in FIG. 1, the first oxygen saturation degree observation filter Fs1 has spectral characteristics that allow light in the wavelength region from the isosbestic points E1 to E4 (i.e., the wavelength region R0) to pass with low loss, and block light in other wavelength regions. The second oxygen saturation degree observation filter Fs2 has spectral characteristics that allow light in the wavelength region from the isosbestic points E2 to E3 (i.e., the wavelength region R2) to pass with low loss, and block light in other wavelength regions.

Note that a through-hole 413 is formed in the peripheral edge portion of the rotating filter 410. The through-hole 413 is formed at a predetermined position (e.g., a position where the through-hole 413 follows a special light filter) in the rotation direction of the rotating filter 410. A photo interrupter 422 for detecting the through-hole 413 is arranged in the periphery of the rotating filter 410 so as to surround a portion of the peripheral edge portion of the rotating filter 410. The photo interrupter 422 is connected to the filter control unit 420.

The endoscope system 1 according to the present embodiment has two operating modes, namely a normal observation mode and a spectral analysis (special observation) mode. The normal observation mode is an operating mode for capturing color images using normal light. The spectral analysis mode is a mode for performing spectral analysis based on digital image data obtained using illumination light IL (special light) that passes through the special light filters Fs1 and Fs2 respectively, and displaying a biomolecule distribution image of biological tissue (e.g., a degree of oxygen saturation distribution image). The operating mode of the endoscope system 1 is switched by a user operation performed on an operation panel (not shown) of the processor 200 or an operation button (not shown) of the electronic endoscope 100, for example.

In the normal observation mode, the controller 530 controls the shift drive mechanism 470 to shift the rotating filter 410 from the application position to the retracted position. Note that in the spectroscopic analysis mode, the rotating filter 410 is arranged at the application position. Then, digital image data obtained by the image sensor 141 is subjected to predetermined image processing such as demosaicing, and then converted into a video signal and displayed on the screen of the monitor 300.

In the spectral analysis mode, the controller 530 controls the filter control unit 420, which includes a servo motor (not shown), to drive the rotating filter 410 to rotate at a constant rotational frequency and successively capture images of the biological tissue T using illumination light IL that passes through the special light filter Fs1, the slit SL1, the special light filter Fs2, and the slit SL2. Then, the controller 530 generates an image showing the distribution of biomolecules in biological tissue, based on digital image data obtained using illumination light IL that passes through the special light filters Fs1 and Fs2, generates a display screen in which the image and the normal observation image obtained using the slits SL1 and SL2 are arranged side-by-side, and furthermore converts the display screen into a video signal and displays the video signal on the monitor 300.

In the spectroscopic analysis mode, the filter control unit 420 detects the phase of rotation of the rotating filter 410 based on the timing of detection of the through-hole 413 by the photo interrupter 422, compares the detected phase with the phase of a timing signal supplied by the controller 530, and adjusts the phase of rotation of the rotating filter 410. The timing signal from the controller 530 is synchronized with the drive signal for the image sensor 141. Accordingly, the rotating filter 410 is driven to rotate at a substantially constant rotational frequency in synchronization with the driving of the image sensor 141. Specifically, the rotation of the rotating filter 410 is controlled such that the one of the special light filter Fs1, the slit SL1, the special light filter Fs2, and the slit SL2 that white light WL enters is switched each time one image (three R, G, and B frames) is captured by the image sensor 141. The filter control unit 420, which includes a servo motor, functions as a rotation drive unit that successively inserts the slits (SL1 and SL2) and the special light filters (Fs1 and Fs2) of the rotating filter 410 into the optical path of white light from the light source 430.

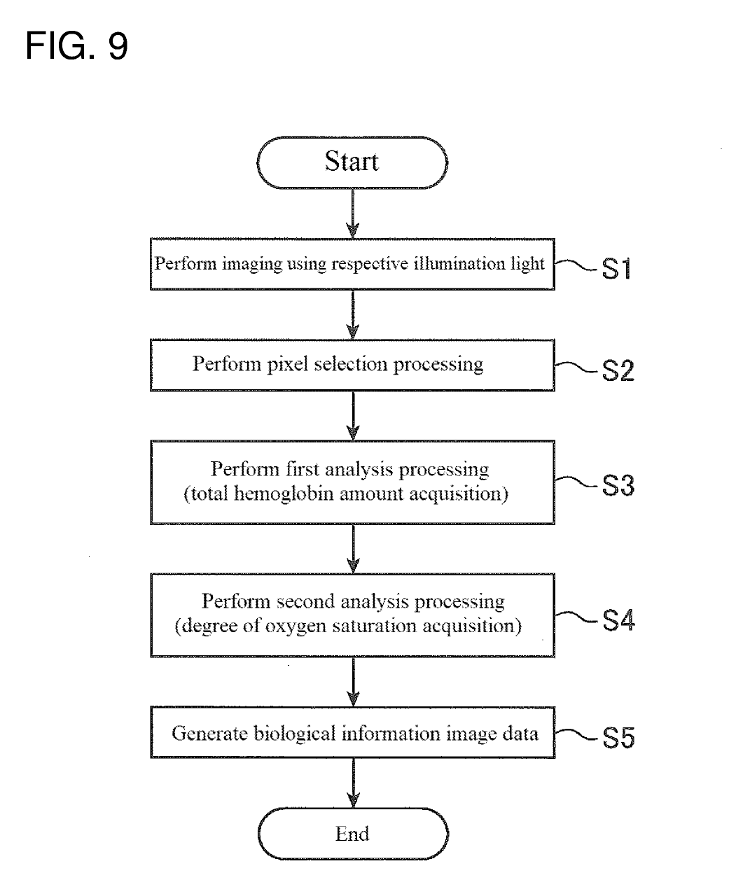

Next, spectral analysis processing executed in the spectral analysis mode will be described. FIG. 9 is a flowchart showing a procedure of spectral analysis processing.

If the spectral analysis mode has been selected by a user operation, the filter control unit 420 drives the rotating filter 410 to rotate at a constant rotational frequency as described above. Then, illumination light IL that passes through the special light filter Fs1, the slit SL1, the special light filter Fs2, and the slit SL2 is successively supplied from the light source unit 400, and images are successively captured using the respective types of illumination light IL (S1). Specifically, G digital image data W(x,y) obtained using illumination light IL that passes through the special light filter Fs1, G digital image data N(x,y) obtained using illumination light IL that passes through the special light filter Fs2, and R digital image data R(x,y), G digital image data G (x,y), and B digital image data B (x,y) obtained using illumination light IL that passes through slits SL1 and SL2 (white light) are stored in the internal memory 532 of the controller 530.

Next, the image processing unit 500 performs pixel selection processing S2 for selecting pixels that are to be subjected to subsequent analysis processing (processing S3-S8), using the R digital image data R (x,y), the G digital image data G (x,y), and the B digital image data B (x,y) acquired in processing S1.

At locations where blood is not included, or locations where the biological tissue color is dominantly influenced by a substance other than hemoglobin, even if the degree of oxygen saturation Sat or blood flow is calculated based on color information of the pixel, a meaningful value is not obtained, but rather is simply noise. If such noise is presented to a physician, it will not only be a hindrance to the physician's diagnosis, but also have the harmful effect of placing an unnecessary burden on the image processing unit 500 and reducing the processing speed. In view of this, the analysis processing of the present embodiment is configured such that pixels suited to analysis processing (i.e., pixels recording the spectroscopic features of hemoglobin) are selected, and analysis processing is performed on only the selected pixels.

In pixel selection processing S2, only pixels that satisfy all of the conditions of Expressions 4, 5, and 6 below are selected as target pixels for analysis processing.

B(x,y)/G(x,y)>a₁ Expression 4

R(x,y)/G(x,y)>a₂ Expression 5

R(x,y)/B(x,y)>a₃ Expression 6

Here, a1, a2, and a3 are positive constants.

The above three conditional expressions are set based on the magnitude relationship of G component value<B component value<R component value in the transmission spectrum of blood. Note that pixel selection processing S2 may be performed using only one or two of the above three conditional expressions e.g., using only Expressions 5 and 6 when focusing on the color red which is specific to blood.

Next, the image processing unit 500 performs first analysis processing S3. The nonvolatile memory 532 of the controller 530 holds the numerical value table T1 (or function) that expresses the quantitative relationship between the total hemoglobin amount tHb and the parameter W/R shown in the graphs (D1) and (D2) in FIG. 4. In the first analysis processing S3, this numerical value table T1 is used to acquire the value of the total hemoglobin amount tHb based on the G digital image data W(x,y) and the R digital image data R(x,y) acquired in processing S1.

Specifically, first, the parameter W/R(x,y) for each pixel (x,y) is calculated using Expression 7.

W/R(x,y)=W(x,y)/R(x,y) Expression 7

Next, the numerical value table T1 is referenced to read out and acquire the value of the total hemoglobin amount tHb(x,y) that corresponds to the value of the parameter W/R(x,y) calculated using Expression 7.

The quantitative relationship in the numerical value table T1 (and the later-described numerical value table T2) held in the nonvolatile memory 532 is obtained in advance by theoretical calculation or experimentation. Note that although a complete one-to-one correspondence does not exist for the value of the total hemoglobin amount tHb and the value of the parameter W/R in the graphs (D1) and (D2), a representative one-to-one quantitative relationship (e.g., average value or mean value) is held in the numerical value table T1 for the total hemoglobin amount tHb and the parameter W/R. For this reason, the total hemoglobin amount tHb can be uniquely determined based on the value of the parameter W/R using the numerical value table T1.

Next, the image processing unit 500 performs second analysis processing S4. The nonvolatile memory 532 of the controller 530 holds the numerical value table T2 (or function) that expresses the quantitative relationship between the total hemoglobin amount tHb, the parameter N/W, and the degree of oxygen saturation Sat shown in the graph (F2) in FIG. 4. Three numerical values (called a “numerical value set”), namely the total hemoglobin amount tHb, the parameter N/W, and the degree of oxygen saturation Sat, are registered in association with each other in the numerical value table T2. In the second analysis processing S4, this numerical value table T2 is used to acquire the value of the degree of oxygen saturation Sat(x,y) for each pixel based on the G digital image data W(x,y) and N(x,y) acquired in processing S1 and the value of the total hemoglobin amount tHb(x,y) acquired in first analysis processing S3.

Specifically, first, the parameter N/W(x,y) for each pixel (x,y) is calculated using Expression 8.

N/W(x,y)=N(x,y)/W(x,y) Expression 8

Next, for each pixel (x,y), the numerical value table T2 is referenced to extract the numerical value set that is closest to the value of the total hemoglobin amount tHb(x,y) acquired in first analysis processing S3 and the value of the parameter N/W(x,y) calculated using Expression 8, and then the value of the degree of oxygen saturation Sat in the extracted numerical value set is read out and acquired as the value of the degree of oxygen saturation Sat(x,y) at that pixel (x,y).

The nonvolatile memory 532 of the controller 530 stores a numerical value table (or function) that expresses the relationship between the degree of oxygen saturation Sat(x,y) and display colors (pixel values). Then, in processing S5 (FIG. 6), the controller 530 references this numerical value table (or function), acquires values that indicate the display colors corresponding to the degree of oxygen saturation Sat(x,y) obtained in processing S4, and generates degree of oxygen saturation distribution image data using these values as pixel values.

The controller 530 then generates normal observation image data based on the R digital image data R(x,y), the G digital image data G(x,y), and the B digital image data B(x,y) that were obtained using illumination light IL (white light) that passes through the slit SL1 (or SL2).

The controller 530 then uses the generated degree of oxygen saturation distribution image data and normal observation image data to generate screen data in which the normal observation image and the degree of oxygen saturation distribution image are displayed side-by-side in one screen, and stores the screen data in the video memory 540. Note that in accordance with a user operation, the controller 530 can generate various types of display screens, such as a display screen that displays only the degree of oxygen saturation distribution image, a display screen that displays only the normal observation image, or a display screen that displays supplementary information such as patient ID information and observation conditions in a superimposed manner on the degree of oxygen saturation distribution image and/or the normal observation image.

Malignant tumor tissue has a higher total hemoglobin amount than normal tissue due to angiogenesis, and also exhibits remarkable oxygen metabolism, and therefore it is known that the degree of oxygen saturation is lower than that of normal tissue. In view of this, the controller 530 can extract the pixels for which the total hemoglobin amount acquired by first analysis processing S3 is greater than a predetermined reference value (first reference value), and for which the degree of oxygen saturation acquired by second analysis processing S4 is less than a predetermined reference value (second reference value), perform enhanced display processing on corresponding pixels of normal observation image data for example to generate enhanced lesion site image data, and display the enhanced lesion site image on the monitor 300 along with the normal observation image and/or the degree of oxygen saturation distribution image (or on its own).

Examples of enhanced display processing include processing for increasing the pixel values of corresponding pixels, processing for changing the hue (e.g., processing for increasing the redness by increasing the R component, or processing for rotating the hue by a predetermined angle), and processing for flashing corresponding pixels (or periodically changing the hue).

Also, a configuration is possible in which, instead of generating enhanced lesion site image data, the controller 530 calculates an indicator Z(x,y) that indicates the degree of suspicion of a malignant tumor based on the deviation of the degree of oxygen saturation Sat(x,y) from an average value and the deviation of the total hemoglobin amount tHb(x,y) from an average value, and generate image data in which the pixel values are the indicator Z (malignancy suspicion image data).

In this way, according to one embodiment, it is preferable that the controller 530 generates information that indicates the state of biological tissue, based on the ratio W/R of the value of R digital image data, which is a color component included in image data obtained by imaging the biological tissue illuminated by white light, and the value of G digital image data included image data obtained by imaging the biological tissue illuminated by special light.

### Rotating Filter Shift Mechanism

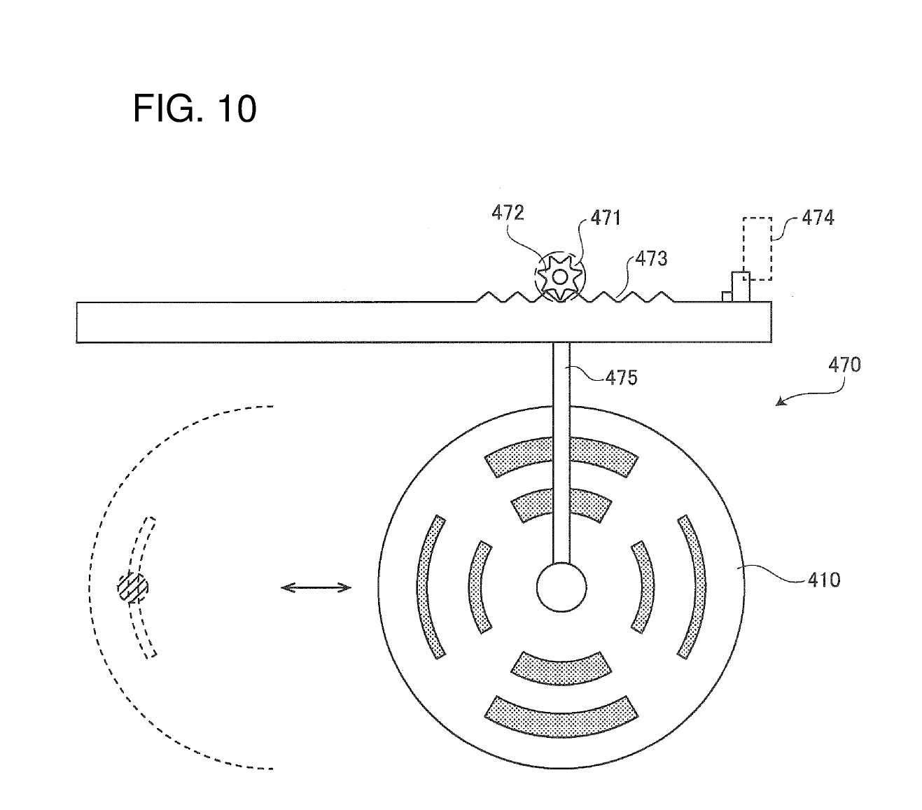

Next, the configuration of the shift drive mechanism (shift drive unit) 470 will be described. FIG. 10 is a diagram schematically showing an example of the configuration of the shift drive mechanism 470 according to an embodiment. Note that FIG. 10 shows the configuration of the shift drive mechanism 470 seen from the condensing lens 450 side. As shown in FIG. 10, the shift drive mechanism 470 includes, for example, a stepping motor 471, a pinion gear 472 that is connected to the drive shaft of the stepping motor 471 via a gear mechanism (not shown), a rack gear 473, an arm 475, and a photo interrupter 474 that detects the origin position of the rotating filter 410. The arm 475 fixes the rotating filter 410 to the rack gear 473. With the above-described configuration of the shift drive mechanism 470, it is possible to perform control to retract the rotating filter 410 from the optical path of illumination light, to position the special light filters and slits located on the outer circumference side on the optical path of illumination light, position the special light filters and slits located on the inner circumference side on the optical path of illumination light, and so on, to generate special light observation images that are suitable for the purpose of observation. The controller 530 moves and stops the rotating filter 410 at a position corresponding to the purpose of observation according to an operation input by the operator via an operation panel.

Problem Arising from Variation of Brightness Corresponding to Variation of Stop Position of Rotating Filter

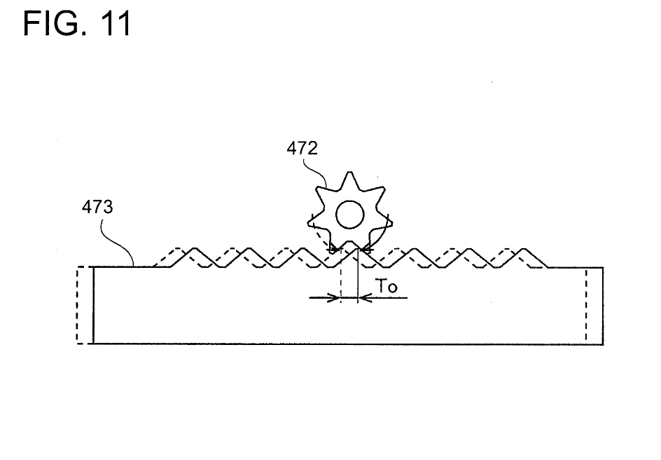

As described above, in a configuration in which the mechanical mechanism moves and stops the rotating filter 410, the position at which the rotating filter 410 stops may vary due to manufacturing tolerances of the mechanical mechanism. Manufacturing tolerances involve various mechanical factors such as a motor backlash, a gear mating error, and so on. FIG. 11 is a diagram illustrating variation of the stop position of the rotating filter 410 that occurs due to such manufacturing tolerances. As shown in FIG. 11, even if the stepping motor 471 is controlled at the position specified by the same step number, an error T₀, which is shown in FIG. 11, occurs in regard to the stop position of the rack gear 473, depending on the rotation direction of the pinion gear 272. It is envisioned that factors that cause variation of the stop position of the rotating filter 410, which is dependent on the rotation direction of the pinion gear, are a combination of various factors such as a mechanical error of the rotating filter 410, a mechanical error in position of the photo interrupter 474, and so on, in addition to the above-described factors.

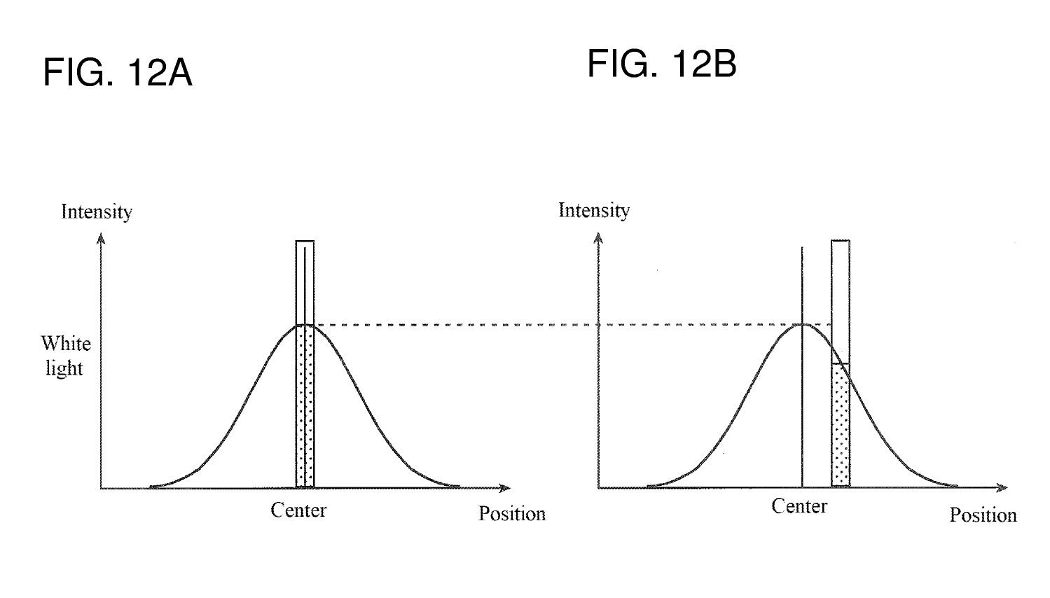

The following describes a problem arising when the stop position of the rotating filter 410 varies in this way. As shown in FIG. 12, it is envisioned that the distribution of intensities of white light emitted from the light source 430 and entering the rotating filter 410 has a peak in a central portion and the intensity gradually decreases from a peak region to the outer sides. If a configuration in which white light passes through slit-shaped opening portions is employed as in the present embodiment, there is a difference in the amount of illumination light IL (normal light) between when a slit (SL1, for example) is located at the center of white light (FIG. 12(a)) and when a slit (SL1, for example) is displaced from the center of white light (FIG. 12(b)). Note that the shaded areas in FIG. 12 correspond to a slit (SL1, for example). Generally, the calculation of biological information (evaluation values) such as the above-described degree of oxygen saturation and the simultaneous display of a normal light image and a special light image, for example, are performed assuming that a slit (SL1, for example) is positioned in the peak region of white light. Therefore, if the position of a slit (SL1, for example) varies as shown in FIG. 12(b) and the amount of normal light is smaller than an expected amount, there is the possibility of a problem occurring, e.g., such a variation affects accuracy in calculating the biological information (evaluation values), or lowers the brightness of a normal light observation image displayed at the same time as a special light observation image.

Therefore, according to an embodiment, the cross section of the luminous flux of white light when entering the slits (SL1, SL2, and so on) and the special light filters (Fs1, Fs2, and so on) is larger than the incident surfaces of the slits (SL1, SL2, and so on) and the special light filters (Fs1, Fs2, and so on), a portion of the luminous flux of white light enters the slits (SL1, SL2, and so on) and the special light filters (Fs1, Fs2, and so on), and the remaining portion does not enter the slits (SL1, SL2, and so on) or the special light filters (Fs1, Fs2, and so on). In this case, it is preferable that the controller 530 controls the shift drive mechanism 470 such that some of white light that enters at least one of a slit (SL1 or SL2, for example) and a special light filter (Fs1 or Fs2, for example) includes the peak position of the light intensity distribution.

The following describes an example of a configuration in which parameters, which are used to correct the amount of displacement from a target position when the shift drive mechanism 470 shifts the rotating filter 410 from a predetermined position to the target position, are stored in advance, and the shift drive mechanism 470 is controlled based on these parameters such that a slit (SL1, for example) is positioned within a predetermined peak position range where the intensity of white light is at its maximum, in other words, the shift drive mechanism 470 is controlled such that a slit is positioned with reference to the peak position at which the intensity of white light is at its maximum, and thus the above-described problem is solved.

### Rotating Filter Stop Position Control 1

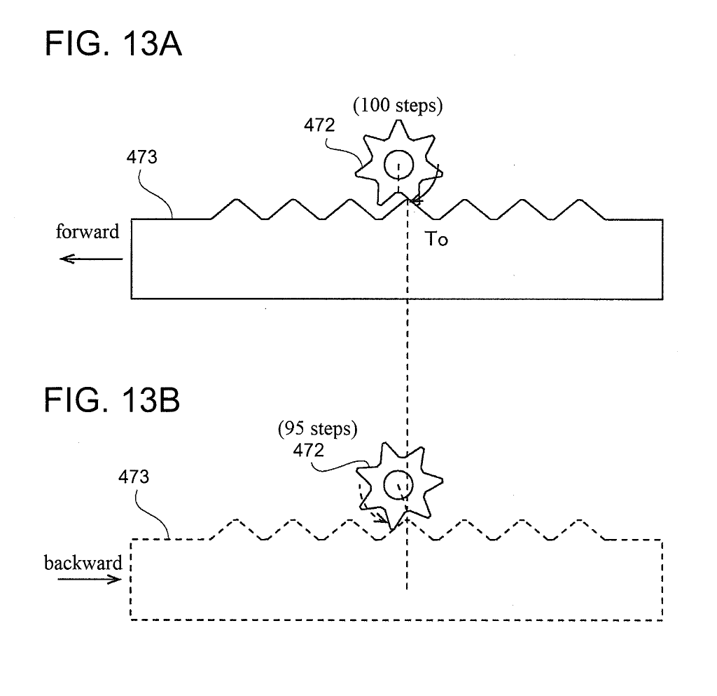

Variation of the stop position of the rack gear 473 shown in FIG. 11, which is dependent on the rotation direction of the pinion gear 472, occurs because there is a difference between the stop position of the rack gear 273 in the forward operation (the rotating filter 410 moves in a direction from the retracted position to enter the optical path) and that in the backward operation (the rotating filter 410 moves in a direction from the optical path to the retracted position). Therefore, it is possible to solve the problem by controlling the stop position of the rack gear 273 such that there is a difference in the step number of the stepping motor 471 between when the operation in the forward direction is performed and when the operation in the backward direction is performed. According to an embodiment, as shown in FIG. 13(a), if the position at which the rack gear 473 is to be stopped when performing the forward operation is specified by the step number “100” of the stepping motor 471, it is preferable that the position at which the rack gear 473 is to be stopped when performing the backward operation is adjusted so as to be specified by the step number “95” of the stepping motor 471 (FIG. 13(b)), and thus the positions of the teeth of the gear when the rack gear 473 stops accurately coincide with those in the case of the forward operation.

To realize the above-described control, according to an embodiment, it is preferable that the controller 531 holds a table in which drive amounts are set, which have been adjusted such that the rotating filter 410 located at a predetermined reference position that is out of the optical path of the light source 430 is moved such that predetermined slits (SL1 and SL2) and special light filters (Fs1 and Fs2) are brought to a target position in the optical path, or predetermined slits (SL3, SL4, or) and special light filters (Fs3 and Fs4) are brought to a target position in the optical path, and the stepping motor 471 is driven and stopped according to the drive amounts. In this case, the adjusted drive amounts are varied so as to correspond to the shift directions of the rotating filter 410, i.e., so as to correspond to the forward operation and the backward operation of the stepping motor 471.

For example, the drive amounts specify the step number of the stepping motor 471. As shown in Table 2 below, it is preferable that a table of step number adjustment values of the stepping motor 471 is held in the internal memory 532, and control is performed to stop the rack gear 473 at a position specified by a step number shown in this adjustment value table. In the example shown in table 2, in the case where the rack gear 473 is to be stopped in the forward operation, the step number specifying the retracted position is set to 5, and when the special light filters and the slits on the outer circumference side (simply denoted as “Filter 1” in Table 2) are used, the step number is set to 100, and when the special light filters and the slits on the inner circumference side (simply denoted as “Filter 2” in Table 2) are used, the step number is set to 200. On the other hand, in the case where the rack gear 473 is to be stopped in the backward operation, the step number specifying the retracted position is set to 0, and when the special light filters and the slits on the outer circumference side (simply denoted as “Filter 1” in Table 2) are used, the step number is set to 95, and when the special light filters and the slits on the inner circumference side (simply denoted as “Filter 2” in Table 2) are used, the step number is set to 195. For example, the step number is a pulse number that is used for driving the stepping motor 471.

TABLE 2

| Transition Position | Forward Operation | Backward Operation |
|---|---|---|
| Retraction | 5 | 0 |
| Filter 1 | 100 | 95 |
| Filter 2 | 200 | 195 |

Regardless of which special light filter or slit is used, it is possible to match the position of the slit of the rotating filter 410 with the peak position of illumination light, or bring the slit within a predetermined range of the peak position, by controlling the stop position of the rack gear 473 using the adjustment values shown in the above Table 2. Note that Table 2 shows examples of adjustment values in a case where the rotating filter 410 has two pairs of special light filters and two pairs of slits, each arranged in a radial direction. However, if the rotating filter 410 has more special light filters or slits arranged in a radial direction, the number of adjustment values to be held can be increased according to the number of pairs of special light filters or slits.

Also, considering changes over time in tolerances of mechanical mechanism, the above-described adjustment value table may be updated. Specifically, the controller 530 has the function of accumulating the usage time of the processor 200 based on the internal clock, and therefore the controller 530 may update the adjustment values by one step per year, for example, using this accumulation function. Table 3 shown below is an example of an adjustment value table that is to be applied one year later, assuming that a configuration in which the adjustment values are updated by one step per year is employed.

TABLE 3

| Transition Position | Forward Operation | Backward Operation |
|---|---|---|
| Retraction | 6 | −1 |
| Filter 1 | 101 | 94 |
| Filter 2 | 202 | 194 |

The rate of changes (degradation) over time may be determined with reference to values obtained through an endurance test. By employing a configuration in which adjustment values are updated considering changes over time in this way, it is possible to absorb play that increases due to the gear wearing down every time it is used, and maintain accuracy regarding the transition position of the rack gear 473 throughout the lifetime of the product.

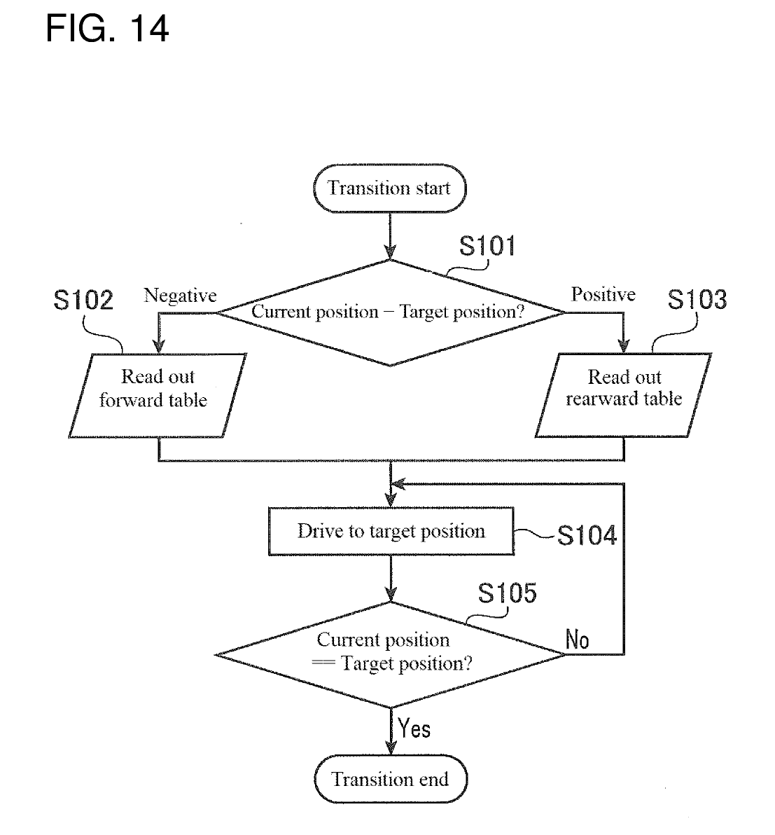

FIG. 14 is a flowchart showing control that is performed when the rotating filter 410 is to be shifted from the current position to the target position using the above-described adjustment value table. Note that the control shown in FIG. 14 is, for example, performed under the control of the controller 530 in response to an operation that is performed by the operator operating an operation panel (not shown) of the processor to shift the position of the rotating filter 410 (an operation performed to carry out a desired special light observation. Upon this processing being started, first, the difference between the current position of the rotating filter 410 and the target position is calculated, and whether the shift direction of the rotating filter 410 is the forward direction or the backward direction is determined (step S101). In step S1, if the result of the calculation is negative, which means that the shift direction of the rotating filter 410 is the forward direction (S101: NEGATIVE), a step number is read out from the “Forward Operation” field of the adjustment value table (Table 2) (step S102). Then, the stepping motor 471 starts driving (step S104), using the step number read out in step S2, and this driving operation using the step number continues until the rotating filter 410 reaches the target position (S105: NO).

Information regarding the current position can be obtained by the controller 530 using the drive amount supplied to the above-described stepping motor 471. Information regarding the target position can be obtained by the controller 530, based on the type of special light that has been set to be used in the spectral analysis mode. According to an embodiment, it is also preferable that the difference between the current position and the target position is calculated based on the difference between the actual amount of white light emitted from the rotating filter 410, obtained through measurement, and a predetermined reference light amount. It is also possible to calculate the difference between the current position and the target position based on the difference between the brightness of the current image captured by the electronic endoscope system 1 and the brightness of an ideal reference image, in order to determine a slit or a special filter at which position on the rotating filter 410 is to be used in the spectral analysis mode.

On the other hand, if the result of the calculation performed in step S101 is positive, which means that the shift direction of the rotating filter 410 is the backward direction (S101: POSITIVE), a step number is read out from the “Backward Operation” field of the adjustment value table (Table 2) (step S103). Then, the stepping motor 471 starts driving (step S104), using the step number read out in step S102, and this driving operation continues until the rotating filter 410 reaches the target position (S105: NO). Upon the rotating filter 410 reaching the target position (S105: YES), this control ends.

In this way, it is preferable that the controller 530 holds, in advance, parameters, such as step numbers, for correcting the amount of displacement that, when the controller 530 controls the shift drive mechanism 470 to shift the rotating filter 410 from a first position to a second position that is the target position, occurs between an actual position to which the rotating filter 410 is shifted and the second position, due to apparatus manufacturing errors or tolerances of the shift drive mechanism 470, and controls the shift drive mechanism 470 based on the parameters.

It is also preferable that the controller 530 changes the drive amount of the shift drive mechanism 470 according to the shift direction of the rotating filter 410 when shifting the rotating filter 410 between the first position and the second position using the shift drive mechanism 470.

Through the above-described position control, it is possible to achieve accurate control of the position of the rotating filter 410 using the adjustment value table.

### Rotating Filter Stop Position Control 2

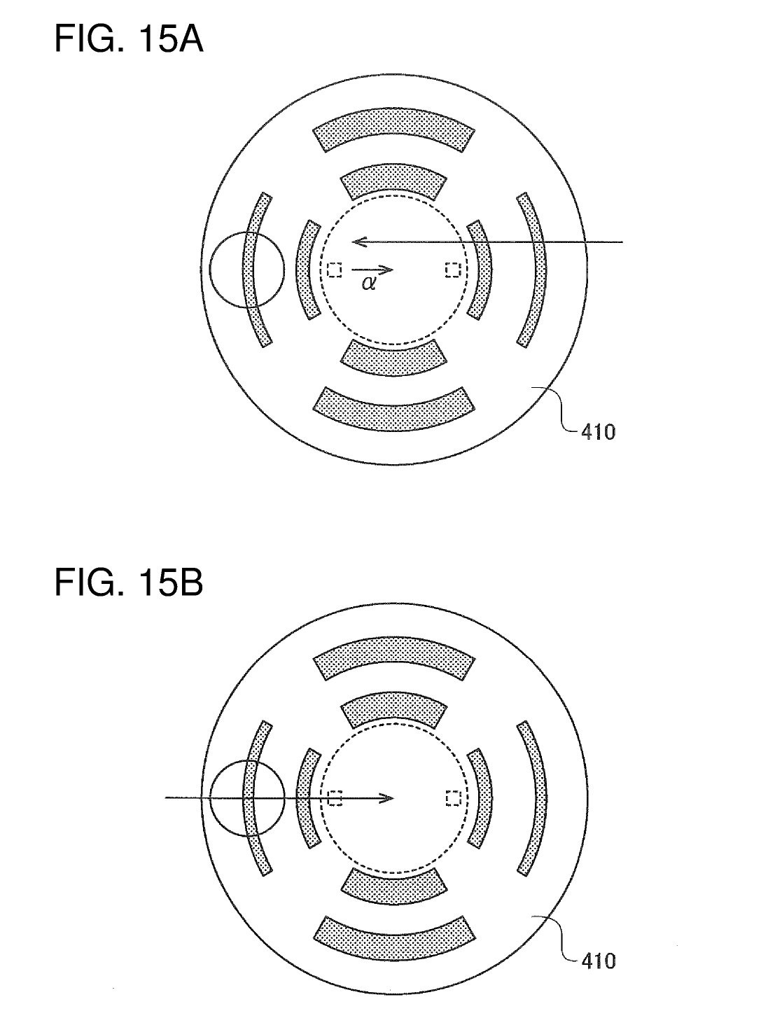

Next, another embodiment of stop position control for the rotating filter 410 will be described. In the stop position control 2, unlike in the above-described stop control 1, the adjustment value table is not used, and the rotating filter 410 is controlled so as to approach the target position by invariably moving in either one of the forward direction or the backward direction and stop at the target position. FIG. 15 shows an operating principle of this control. Here, there is the presumption that the step number of the stepping motor correctly corresponds to the target position when the rotating filter 410 performs the backward operation, for example.

As shown in FIG. 15(a), according to an embodiment, when the shift direction from the current position to the target position is the forward direction, the rotating filter 410 is temporarily moved to a position beyond the target position, and is then moved back to the target position through the backward operation. Here, the rotating filter 410 is moved beyond the target position by a passing amount α, for example. On the other hand, as shown in FIG. 15(b), when the shift direction from the current position to the target position is the backward direction, the rotating filter 410 is moved directly to the target position. Through such control, it is possible to allow the rotating filter 410 to invariably approach and stop at the target position through the backward operation.

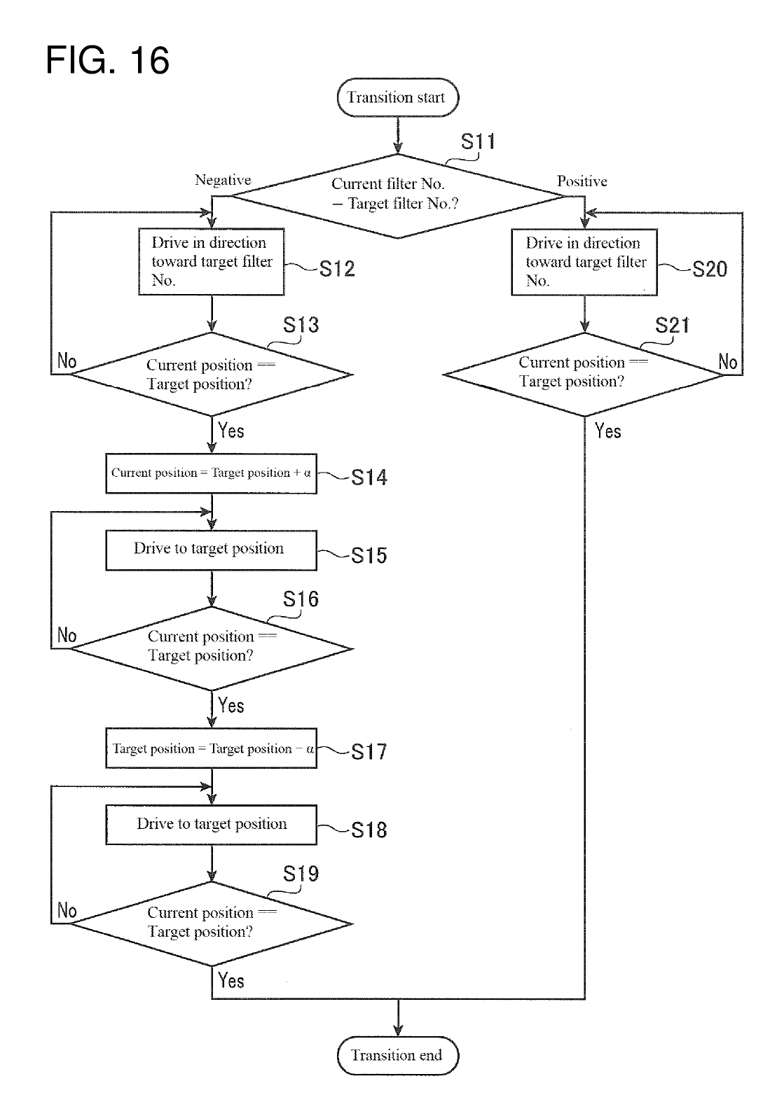

FIG. 16 is a flowchart for realizing the above-described control. In this example, when the rotating filter 410 is provided with a plurality of pairs of special light filters arranged in the radial direction, numbers are sequentially assigned to the special light filters in ascending order from the outer side (i.e., filter numbers, such as filter 1, filter 2, filter 3, . . . , are sequentially assigned in ascending order from the outer side). The control shown in FIG. 16 is performed under the control of a control signal generated by the controller 530 in response to the operator performing an operation on an operation panel (not shown) of the processor 200 to shift the position of the rotating filter 410 (an operation performed to carry out a desired special light observation).

Upon this processing being started, first, processing is performed to subtract the target filter number from the filter number of the filter at the current position (step S11). If the result of the reduction processing performed in step S11 is negative, the rotating filter 410 is to be shifted in the forward direction, and therefore processing proceeds to step S12 and the rotating filter 410 starts being driven to move to the target filter number. This driving continues until the rotating filter 410 is shifted to the position specified by the target filter number (step S13: NO). Upon the rotating filter 410 reaching the target position (step S13: YES), processing is performed to add the passing amount a to the target position (step S14), and the driving is further continued (step S15). This driving continues until the rotating filter 410 reaches the target position set in step S15 (step S16: NO).

Upon the rotating filter 410 reaching the target position set in step S15 (step S16: YES), processing is performed next to subtract the passing amount a from the target position (step S17). Then, processing is performed to drive and move the rotating filter 410 to the target position (step S18). This driving continues until the rotating filter 410 reaches the target position set in step S17 (step S19: NO). Upon the rotating filter 410 reaching the target position set in step S17 (step S19: YES), this control processing ends.

On the other hand, if the result of the calculation performed in step S11 is positive, the shift direction of the rotating filter 410 is the backward direction (step S11: POSITIVE), processing proceeds to step S20. In step S20, the rotating filter 410 is started being driven to move to the target position, and this driving continues until the rotating filter 410 reaches the target position (step S21: NO). Upon the rotating filter 410 reaching the target position (S21: YES), this processing ends.

Through the above-described position control processing, it is possible to allow the rotating filter 410 to invariably approach and stop at the target position through the backward operation, and to accurately stop at the target position. Note that the position control processing shown in FIG. 16 is based on the premise that the step number of the stepping motor accurately corresponds to the target position when the rotating filter 410 performs the backward operation. If there is the premise that the step number of the stepping motor accurately corresponds to the target position when the rotating filter 410 performs the forward operation, processing that is similar to the above-described processing, which uses the passing amount, is performed in the series of processing for the case where the result of the calculation performed in step S11 is positive in the flowchart shown in FIG. 16.

That is, it is preferable that the controller 530 controls the shift drive mechanism 470 such that the shift direction when a slit (SL1 or SL2, for example) of the rotating filter 410 is caused to enter and stop in the optical path of white light by the shift drive mechanism 470, i.e., the shift direction immediately before the rotating filter 410 stops, is invariably constant. Preferably, at this time, when shifting the rotating filter 410 from the first position to the second position, the controller 530 shifts the rotating filter 410 beyond the second position from the first position, and thereafter reverses the shift direction and shifts the rotating filter 410 to the second position.

Although it is preferable that the passing amount a is as small as possible from the viewpoint of quickly shifting the rotating filter 410, it may be set to a necessary and sufficient amount, considering various tolerances such as those shown below.
A tolerance for the working accuracy of a rotary turret that constitutes the rotating filter (e.g., 0.1 mm).
Tolerances for the working accuracy of all of the constituent gears, and the amount of play between meshing gears (e.g. 1.0 mm).
The amount of play assumed for aging/durability, etc. (e.g. 1.0 mm).
Any safety ratio.

Hereinafter, effects that are produced by the above-described embodiment will be further described. As described above, it is possible to perform control such that the position of a slit (SL1 for example) of the rotating filter 410 is within a predetermined range of the peak position of white light. According to an embodiment, the range of the shift amount (i.e., the amount of shift of the stop position of the rotating filter) with reference to a luminous flux diameter (the diameter of the luminous flux that enters the rotating filter) is preferably 0% to 4%, more preferably 0% to 2%, and further preferably 0% to 1%. Note that, in order to reduce the shift amount to zero, it is necessary to improve the various kinds of accuracy, which may increase costs. Therefore, the lower limit of the shift amount may be 0.1% or more, or 0.3% or more. Note that, according to an embodiment, it is possible to reduce the shift amount with reference to the luminous flux diameter to 0.7% under specific conditions (it is envisioned that the shift amount in this case includes almost no gear tolerances or the like, and is the remaining shift amount caused by another factor). In a conventional configuration, which is a comparative example, the shift amount with reference to the luminous flux diameter is approximately 5% (in this case, factors such as gear tolerances or the like are dominant). Note that the effects described above are those in the case where the luminous flux diameter is 10 mm, and the effects of each embodiment are not limited to the examples of numeral values shown above.

### Ratio Adjusting Function

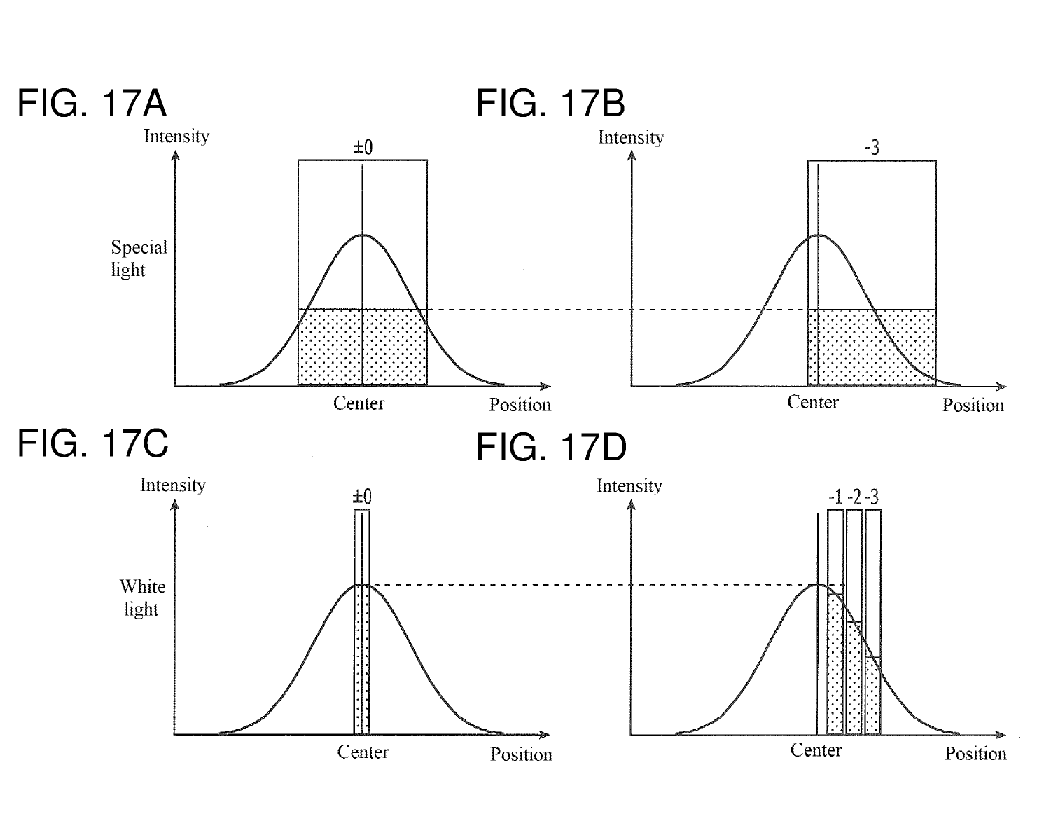

The above-described configuration makes it possible to accurately control the positions of the slits (SL1 and SL2) of the rotating filter 410, using the shift drive mechanism 470. In a state where a normal observation image and a special light observation image are displayed at the same time, it is possible to adjust the brightness ratio of the normal observation image by using the above-described configuration. FIG. 17 is a diagram for illustrating a principle of this fact. First, it is envisioned that the shift drive mechanism 470 is controlled such that a slit is accurately positioned in the optical path from the light source 430. If this is the case, as shown in FIG. 17(c), the slit (SL1 or SL2) is located at the peak position of the intensity distribution of white light (the shaded portion in FIG. 17(c) corresponds to the position of the slit), and an amount of normal light corresponding to the peak position can be obtained. FIG. 17(a) shows a portion of the intensity distribution of white light that a special light filter (Fs1 or Fs2) overlaps in this case. In FIG. 17(a), the shaded portion corresponds to the special light filter (Fs1 or Fs2). As described above, the special light filters Fs1 and Fs2 have a sufficiently large width in the radial direction, compared to the slits (SL1 and SL2). Therefore, it can be understood that the special light filter (Fs1 or Fs2) uses a significantly large area of the intensity distribution of white light in a state shown in FIG. 17(a).

Here, as shown in FIG. 17(d), it is envisioned that a slit (SL1 or S12) of the rotating filter 410 is moved stepwise from the peak position of the white light, a predetermined amount at a time, by driving and controlling the shift drive mechanism 470. If this is the case, as shown in FIG. 17(d), it is possible to obtain an amount of light that decreases stepwise, using the slit (SL1 or SL2). The example in FIG. 17(d) shows a state in which three kinds of light amounts that decrease stepwise (−1 level, −2 level, and −3 level) can be obtained. In contrast, in this case, the special light filter (Fs1 or Fs2) has a sufficient width in the radial direction, and therefore, even if the rotating filter 410 is moved to “−3 level” shown in FIG. 17(d), a decrease in the amount of white light is small, and a sufficient amount of white light is allowed to pass through the rotating filter 410. Thus, the emission intensity of illumination light from the special light filter (Fs1 or Fs2) does not decrease to the extent that the brightness of an image is substantially influenced.

Therefore, by accurately moving the position of the rotating filter 41 stepwise a small distance at a time according to the above-described driving example, when displaying a normal observation image and a special observation image at the same time, it is possible to adjust the brightness ratio of the normal observation.

As described above, with the endoscope system 1, the parameter W/R for calculating the total hemoglobin amount tHb is obtained according to the spectral analysis mode shown in FIG. 9, using special light and white light as illumination light for illuminating the subject. The value of the parameter W/R is an important value for calculating the total hemoglobin amount tHb and further calculating the degree of oxygen saturation Sat from the total hemoglobin amount tHb thus calculated. The value of the parameter W/R is the ratio between the light intensity of the white light component that passes through a slit (SL1 or SL2, for example) and the light intensity of the special light component that passes through a special light filter (Fs, Fs2, or the like). Therefore, it is undesirable that the value of the parameter W/R changes when the endoscope system 1 is used a plurality of times, from the viewpoint of calculating an accurate total hemoglobin amount tHb and calculating an accurate degree of oxygen saturation Sat. From this viewpoint, significant effect can be produced by controlling the shift drive mechanism 470 such that the ratio between the amount of special light and the amount of white light is within the reference range.

The wavelength band of special light is narrower than the wavelength band of white light, and the width, in the radial direction of the rotating filter 410, of the special filters (Fs1, Fs2, and so on) that extract special light is larger than the width of the slits (SL1, SL2, and so on) in the radial direction. Thus, the total amount of special light in the overall wavelength band thereof is close to the total amount of white light in the overall wavelength band thereof. Therefore, it is possible to improve the SN ratio of the parameter W/R. As a result, it is possible to obtain an accurate total hemoglobin amount tHb, which leads to an accurate degree of oxygen saturation Sat.

Although an embodiment of the present invention and specific examples of the embodiment been described above, the present invention is not limited to the above configurations, and various modifications can be made within the scope of the technical idea of the present invention.

For example, the configuration of the rotating filter described with reference to FIG. 8 is an example, and the types of special light filters and the number of filters arranged in the radial direction can be variously modified.

In the embodiment above, the white light passing regions of the rotating filter are formed as slits. If the white light passing regions are configured to attenuate white light from the light source, and the above-described problem arises due to the relationship between the peak position of the light intensity of the luminous flux of white light from the light source and manufacturing tolerances of the shift drive mechanism, the configuration of the above-described embodiment effectively functions. For example, the white light passing regions may be provided with filters such as light attenuation filters.

Also, in the above-described embodiment, the present invention is applied to the analysis of the concentration distribution of hemoglobin in biological tissue, but the present invention can also be applied to the analysis of the concentration distribution of another biological substance (e.g., a secretion such as a hormone) that changes the color of biological tissue.

Also, the image sensor 141 of the present embodiment is described as an image sensor for color image capturing that includes R, G, and B primary-color color filters on the front side, but there is no limitation to this configuration, and an image sensor for color image capturing that includes Y, Cy, Mg, and G complementary-color color filters for example may be used.

### DESCRIPTION OF REFERENCE SIGNS

1 Endoscope System
100 Electronic Endoscope
110 Insertion Tube
101 Insertion Tube Distal End Portion
121 Objective Optical System
131 Light Guide
131a Distal End Portion
131b Base End Portion
132 Light Distribution Lens
141 Image Sensor
141a Color Filter
142 Cable
200 Processor
300 Monitor
400 Light Source Unit
410 Rotating Filter
420 Filter Control Unit
430 Light Source
440 Condensing Lens
450 Condensing Lens
470 Shift Drive Mechanism
471 Stepping Motor
472 Pinion Gear
473 Rack Gear
474 Photo Interrupter
500 Image Processing Unit
510 A/D Conversion Circuit
520 Temporary Storage Memory
530 Controller
540 Video Memory
550 Signal Processing Circuit

1. An endoscope system comprising:
a light source that emits a first light;
a rotating plate in which a first light transmitting region that allows the first light to pass therethrough, and a second light transmitting region that extracts a second light that is in at least one specific wavelength region from the first light, are arranged in a predetermined direction, the first light transmitting region being configured to reduce a difference in amount between the first light that passes through the first light transmitting region and the second light extracted by the second light transmitting region;
a rotation drive unit that inserts the first light transmitting region and the second light transmitting region one after the other into an optical path of the first light from the light source by rotating the rotating plate;
a shift drive unit that shifts the rotating plate in a direction that intersects the optical path from the light source; and
a control unit that controls the shift drive unit such that a ratio between the amount of light that has passed through the second light transmitting region and the amount of light that has passed through the first light transmitting region is within a reference range.

2. An endoscope system comprising:
a light source that is configured to emit a first light;
a rotating plate that is provided with a first light transmitting region that allows the first light to pass therethrough and a second light transmitting region that extracts a second light that is in at least one specific wavelength region from the first light, and is configured to position the first light transmitting region and the second light transmitting region one after the other in an optical path of the first light to generate the first light and the second light one after another;
a shift drive unit configured to shift the rotating plate in a direction that intersects the optical path of the first light; and
a control unit configured to control the shift drive unit such that a ratio between the amount of light that has passed through the second light transmitting region and the amount of light that has passed through the first light transmitting region is within a reference range.

3. The endoscope system according to claim 1,
wherein the control unit holds, in advance, a parameter for correcting the amount of displacement that is caused by the shift drive unit, between an actual position to which the rotating plate is shifted and a second position when the control unit controls the shift drive unit to shift the rotating plate from a first position to the second position, which is a target position, and controls the shift drive unit based on the parameter.

4. The endoscope system according to claim 1,
wherein the control unit controls the shift drive unit such that a position in the optical path into which the first light transmitting region of the rotating plate is inserted by the rotation drive unit is determined with reference to a peak position at which the light intensity of the first light from the light source is at its maximum.

5. The endoscope system according to claim 1,
wherein the first light has a light intensity distribution,
a cross section of a luminous flux of the first light when entering the first light transmitting region and the second light transmitting region is larger than an incident surface of the first light transmitting region and an incident surface of the second light transmitting region, respectively, a portion of the luminous flux of the first light enters the first light transmitting region and the second light transmitting region, and the remaining portion of the luminous flux does not enter the first light transmitting region or the second light transmitting region, and
the control unit controls the shift drive unit such that a portion of the luminous flux of the first light that enters at least one of the first light transmitting region and the second light transmitting region includes a peak position of the light intensity distribution.

6. The endoscope system according to claim 1,
wherein, when shifting the rotating plate between a first position and a second position by controlling the shift drive unit, the control unit changes a drive amount of the shift drive unit according to a shift direction of the rotating plate.

7. The endoscope system according to claim 1,
wherein the control unit controls the shift drive unit such that a shift direction of the rotating plate is constant when the rotating plate is caused to enter and stop in the optical path by the shift drive unit.

8. The endoscope system according to claim 7,
wherein, when shifting the rotating plate from a first position toward a second position, the control unit shifts the rotating plate beyond the second position from the first position, and thereafter reverses the shift direction of the rotating plate and shifts the rotating plate to the second position.

9. The endoscope system according to claim 1,
wherein the control unit controls the shift drive unit based on information regarding mechanical tolerances of the shift drive unit.

10. The endoscope system according to claim 1,
wherein the rotating plate is configured such that the second light transmitting region and the first light transmitting region have different widths in a radial direction.

11. The endoscope system according to claim 10,
wherein a wavelength band of the second light is narrower than a wavelength band of the first light, and
a width of the second light transmitting region in a radial direction is larger than a width of the first light transmitting region in the radial direction.

12. The endoscope system according to claim 1,
wherein the light source is a lamp that emits white light, which is the first light.

13. The endoscope system according to claim 1,
wherein the control unit generates, based on a ratio between a value of image data of a color component included in image data obtained by imaging biological tissue illuminated by the first light and a value of image data of a color component included in image data obtained by imaging the subject illuminated by the second light, information regarding a state of the biological tissue.
